(19) United States
(12) Patent Application Publication
(10) Pub. No.: US 2009/0203892 A1
(43) Pub. Date: Aug. 13, 2009

(54) Retrotransposon Inhibition in Therapy

(75) Inventors: Enrico Garaci, Roma (IT); Paola Sinibaldi, Roma (IT); Corrado Spadafora, Roma (IT); Carmine Pittoggi, Roma (IT); Ilaria Sciamanna, Roma (IT); Cristina Mearelli, Roma (IT)

Correspondence Address: LERNER, DAVID, LITTENBERG,;KRUMHOLZ & MENTLIK, 600 SOUTH AVENUE WEST, WESTFIELD, NJ, 07090, US

(73) Assignees: ENRICO GARACI, ROMA (IT); PAOLA SINIBALDI, ROMA (IT); ISTITUTO SUPERIORE DI SANITA, ROMA (IT)

(21) Appl. No.: 11/794,554

(22) PCT Filed: Dec. 30, 2005
(86) PCT No.: PCT/EP05/14206

§ 371 Date: Jan. 14, 2008

(30) Foreign Application Priority Data
Dec. 30, 2004 (GB) 0428522.7

(51) Int. Cl. A61K 31/7105; A61K 31/711; C07H 21/02

(52) U.S. Cl. 536/24.5

(57) ABSTRACT

RNA interference is useful in the treatment of cancerous lesions, wherein the RNA recognises a portion of at least one LINE-I repeat element.

### INTRODUCTION

The present invention relates to the use of inhibitors of reverse transcriptase expression in therapy.

In his paper, Spadafora (Cytogenet Genome Res 105:346-350 (2004)) discussed endogenous, non-telomeric reverse transcriptase and its implications in embryogenesis and transformation.

More particularly, reverse transcriptase (RT) is encoded by two classes of repeated genomic elements, namely retrotransposons and endogenous retroviruses. Both of these require RT as an essential component of their machinery.

Retrotransposable elements, such as long interspersed elements (LINEs), have long been considered to be “junk DNA”, and to serve very little purpose other than as leftover DNA that is no longer required, and which has not been deleted from the genome. As long ago as 1971 (Temin, J Natl Cancer Inst 46:56-60), this position was challenged, but the art continues to consider such elements simply as “junk DNA”.

In his paper, Spadafora (supra) reviews the art and demonstrates that the expression of RT-encoding genes is generally repressed in non-pathological, terminally differentiated cells, but is active in very early embryos, germ cells, embryo and tumour tissues, all of which have a high proliferative potential. Blocking of RT in murine embryos arrested their development, and removing the blocking effect did not restart embryogenesis. In cancer cells, proliferation was markedly reduced, and differentiation noted between 48 and 72 hours.

Kuo et al (Biochem and Biophys Res Com 253:566-570 (1998)) identified a 1.7 kb LINE-1 (L1) transcript from the cDNA library of human small-cell lung cancer. They found that this repeated element was ubiquitously expressed in human tissues, both normal (fibroblast and liver) and transformed. In addition, they showed that a sense oligonucleotide, derived from this transcript and incubated with human hepatoma cells, reduced the rate of cell proliferation. The presence of this element in both normal and cancer tissues appeared to associate this repeat element with the general function of cell proliferation. Reduction in cell proliferation was explained by the authors as being a result of the silencing, or functional alteration, of genes involved in control of cell growth, due to mutation. The authors do not suggest that transformation is a reversible event.

By contrast, we have now established that inhibition of the LINE-1 family of retrotransposons is effective to inhibit or block proliferation of cancerous tissue and to stimulate differentiation thereof.

### SUMMARY OF THE INVENTION

Thus, in a first aspect, the present invention provides the use of RNA interference to inhibit unspecialised proliferation of cancerous tissue, wherein the RNA recognises a portion of at least one LINE-1 repeat element.

The present invention, in an alternative aspect, provides the use of RNA interference (RNAi) in the treatment of a cancerous lesion, wherein the RNA recognises a portion of at least one LINE-1 repeat element.

Reduction of RT expression by use of the RNAi of the invention leads to a reduction in proliferation of cancerous tissue, frequently by greater than 50%, with subsequent proliferation being largely accountable for by differentiated growth, at least in treated cells. Thus, the RNAi of the invention serves generally both to reduce proliferation of cancerous tissue, as well as to stimulate differentiation.

It will be appreciated that the RNAi of the invention is specific to LINE-1, and that use thereof avoids having to use a generic non nucleotide RT inhibitor (NNRTI), which blocks all RTs. Indeed, it is surprising that the RNAi of the invention, directed against LINE-1, serves to block or inhibit proliferation of cancerous tissue, given that LINE-1 is only a sub-group of RT-encoding elements.

Presently, only a few (around six to eight) members of the LINE-1 family are recognised as being particularly highly active, and RNAi against any one of these is envisaged by the present invention. Combination therapy using RNAi, wherein each RNA is specific for an individual LINE-1 retrotransposon, is envisaged, but it is preferred to employ RNA against a consensus sequence. The consensus sequence may be for two or more LINE-1 family members, but is preferably for the active members, and may be for more, if more are identified.

Preferably, the RNAi is short interfering RNA (siRNA) or double-stranded RNA (dsRNA).

It is also preferred that the RNA is short hairpin RNA, preferably adapted for, and preferably administered by, means of an siRNA expression vector. Suitable vectors include plasmids of retroviruses as are well known in the art and also discussed herein.

It is generally preferred that the RNA employed in the present invention has a stretch of 10 or more, such as 15, 20, 30, 40 or more nucleotides which are the direct sense equivalent of a region of transcribed LINE-1 DNA. However 21 nucleotides is particularly preferred. The transcribed LINE-1 DNA is preferably selected from a consensus region. However, it is not essential that the stretch of nucleotides be entirely faithful to the selected region of transcribed LINE-1 DNA, provided that the interfering RNA of the invention serves to bind the transcribed RNA from the LINE-1 DNA.

It is, nevertheless, particularly preferred that the stretch of RNA nucleotides from the RNAi is faithful to the corresponding stretch of transcribed DNA from the LINE-1 sequence. The RNAI preferably comprises, and more preferably consists of, a 21 nucleotide sequence which is faithful to the corresponding stretch of transcribed DNA from the LINE-1 sequence.

The RNAi of the present invention may form a looped structure, wherein the loop may be located within the stretch of nucleotides discussed above, in which case the stretch may be interrupted by the loop. This loop may take the form of dsRNA for part of its structure, and may provide a gap of 1, 2 or 3 nucleotides in the stretch of RNA. It is generally preferred that the loop result in no omissions from the stretch of RNA so that the target mRNA is bound along the selected sequence.

The RNAi of the invention may simply be a short sequence capable of binding the corresponding transcribed sequence from the LINE-1 element, or may additionally comprise one or two terminal sequences and/or an internal loop sequence.

It will be appreciated that the sequence selected within LINE-1 should be an open reading frame, and may be selected from ORF1 and ORF2 of the RT, for example. Thus it is preferred that the open reading frame encodes Reverse Transcriptase. This includes any protein having reverse transcription activity.

RNAi therapy may be administered in any convenient manner. In general, it is important to ensure that the RNAi reaches the target cells.

While the RNAi of the invention may be injected directly to the target site in any suitable vehicle, it may also be administered anchored to scaffolds or nanoparticles, for example.

More preferably, RNAi may be administered as plasmids or via retroviruses, for example. Adenoviruses and adeno-associated viral vectors may be employed to distribute the coding sequence for RNAi, preferably in the form of a plasmid. Other similar viruses and retroviruses may also be employed, as well as other such vehicles. In particular, it has been established that the efficacy of delivering the coding sequence, such as a plasmid, to the target site can be increased in the circulation if a permeation factor is employed, such as vascular endothelial growth factor (VEGF).

Another suitable means for delivery is the pSUPER RNAi system kit (www.oligoengine.com).

### BRIEF DESCRIPTION OF THE FIGURES

FIG. 1. Inhibition of proliferation by anti-RT drugs

(A). Cell growth in cultures treated with DMSO (control), nevirapine (NEV) and efavirenz (EFV). Cells were counted and re-plated every 96 h for five cycles. Cells were then cultured in inhibitor-free medium (two cycles). RT inhibitors were then re-added for two cycles. Cell counts are expressed as the % of controls, taken as 100%. Values represent pooled data from three experiments.

(B). Cell cycle profiles in the presence of RT inhibitors for four 96 h-cycles and after drug removal.

FIG. 2. RT inhibitors induce morphological differentiation in melanoma cells.

(A). A-375 cell line cultured in DMSO-(a, b, c), nevirapine-(d, e, f) or efavirenz-(g, h, i). Cultures were observed by phase-contrast microscopy after Wright Giemsa staining (left column), SEM (middle column), and confocal microscopy (right column) after alpha-tubulin (green) and PI staining of nuclei (red).

(B). Melanoma-derived TVM-A12 primary cells. DMSO-(a, b, c) and nevirapine-treated (d, e, f) cells under phase contrast (left column), SEM (middle column), and confocal microscopy (right column). Bar, 20 μm.

FIG. 3. RT inhibitors modulate gene expression in A-375 (Panel A) and PC3 (Panel B) cell lines.

RNA extracted from cells treated with DMSO (ctr), nevirapine (nev) or efavirenz (efv), and after removal of nevirapine (nev/r) or efavirenz (efv/r), was amplified by RT-PCR, blotted and hybridized with internal oligonucleotides. GAPDH was used as an internal standard.

FIG. 4. RNAi to LINE-1 induces morphological differentiation, reduces proliferation and modulates gene expression in A-375 cells.

(A). Structure of a full-length LINE-1 element. The position of the siRNA oligonucleotide is indicated. Arrowheads indicate the positions of primer pairs used for RT-PCR analysis.

(B). Phase-contrast microscopy of A-375 cultures transfected with control (CTR) and LINE-1 siRNA oligonucleotide (L1-i).

(C). Cell growth after transfection with CTR and L1-i oligonucleotides.

(D). Exemplifying gene expression patterns after semi-quantitative RT-PCR of RNA from A-375 cells transfected with CTR or L1-i oligonucleotides. Quantitative variations (expressed as the % of signal in L1-i to signal in CTR transfected cultures) represent the mean from three independent experiments; amplified products were estimated by densitometry of the bands and normalized to the GAPDH signal in the same experiment.

FIG. 5. Efavirenz reduces human tumor growth in nude mice.

The growth of tumors formed by the indicated cell lines was monitored in untreated animals (red) and in animals treated with efavirenz one day (purple) or one week (yellow) after inoculation. The two curves second from top in PC3 and H129 show the growth of PC3- and H69-derived tumors in animals treated starting one day after the inoculation but subjected to treatment discontinuation after 14 days. Curves show the mean value of tumor size in groups of five animals.

FIG. 6. Reduced tumorigenicity of PC3 cells pre-treated with efavirenz.

(A). Growth of tumors formed by untreated or efavirenz pre-treated cells injected in mice that were not treated or were post-treated with efavirenz in vivo.

(B). The outcome of PC3-derived xenografts after the indicated treatments for 30 days (n=20 animals/group).

FIG. 7. Cells infected with pS-L1 exhibit a drastic reduction of proliferation.

pS-L1 infected cells are shown as A375pS-11. The proliferation remained constant for at least 39 days. Non-infected cells (A375, FIG. 7A) maintained a high proliferation rate, and pS-infected cells (A375 pS, FIG. 7B) showed a moderate reduction of proliferation in the first few days after infection, but subsequently resumed quickly a high proliferation rate comparable to that of non-infected cells.

FIG. 8. Tumor growth was markedly reduced in mice inoculated with LINE1-interfered cells as compared controls.

Panel A, shows progression of tumor growth in mice inoculated with A375 pS and with A375 pS-L1i cells. The examples in panel B, show that tumor growth was markedly reduced in mice inoculated with LINE1-interfered cells as compared to those inoculated with control cells.

### DETAILED DESCRIPTION OF THE INVENTION

The LINE-1 elements (L1s) to which the RNAi of the present invention is directed are preferably selected in accordance with the teachings of Brouha et al (2003), which is hereby incorporated by reference.

Active L1s are preferred because if they are active, they are likely to be capable of expressing RT. Thus, the RNAi of the present invention preferably recognises or targets a portion of at least one active L1. Preferably, expression of RT is inhibited by RNAi.

It will be appreciated that the RNAi sequence used will recognise and be capable of binding to the RNA obtainable by transcription from a particular ORF comprised within the target L1, the L1 element being characterised by the fact that it also comprises the preferred sequence discussed herein.

Thus, the L1 sequence is identified by the preferred sequences of the present invention, for instance SEQ ID NO 27, its corresponding DNA sequence, or homologues thereof, as discussed elsewhere, but the L1 sequence is also preferably capable of expressing a protein, preferably RT, the expression of which is inhibited by the RNAi.

The binding of the RNAi sequence to the LINE-1 RNA is preferably under stringent conditions, such as in a buffer containing 50% formamide and 6×SSC.

It is preferred that the sequence used to characterise the L1 element is itself an ORF, part of an ORF, or comprises at least a portion of an ORF. Preferably, the L1 sequence used targeted by the RNAi is comprised within an ORF of said L1 element.

Therefore, where reference is made to a particular DNA sequence that may be preferred, it will be appreciated that this is a sequence used to identify, and is likely contained within, the larger sequence of the L1 element. As such, the identifying sequence of the L1 element need not contain an ORF, provided that the L1 element itself does contain an expressible ORF, the RNA transcribed from the ORF being capable of being bound by the RNAi directed to targeting that particular L1 element.

Thus, the RNAi preferably targets L1 elements comprising this preferred DNA sequence or its corresponding sequence, or homologues thereof.

Examples of ORFs within preferred L1 elements are the sequences corresponding to the primer sequences SEQ ID NOS. 20-25. Thus, the RNAI preferably recognises these SEQ ID NOS or corresponding sequences thereto.

| | |
|---|---|
| More specifically, for ORF 1: | |
| 5′-AGAAATGAGCAAAGCCTCCA-3′; | (SEQ ID NO. 20) |
| 5′-GCCTGGTGGTGACAAAATCT-3′; | (SEQ ID NO. 21) |
| and | |
| 5′-TAAGGGCAGCCAGAGAGAAA-3′: | (SEQ ID NO. 24) |
| for ORF-2: | |
| 5′-TCCAGCAGCACATCAAAAAG-3′; | (SEQ ID NO. 22) |
| 5′-CCAGTTTTTGCCCATTCAGT-3′; | (SEQ ID NO. 23) |
| and | |
| 5′-TGACAAACCCACAGCCAATA-3′. | (SEQ ID NO. 25) |

Accordingly, the RNA equivalents of these sequences are, for ORF1:

| | |
|---|---|
| 5′-AGAAAUGAGCAAAGCCUCCA-3′; | (SEQ ID NO. 39) |
| 5′-GCCUGGUGGUGACAAAAUCU-3′; | (SEQ ID NO. 40) |
| and | |
| 5′-UAAGGGCAGCCAGAGAGAAA-3′: | (SEQ ID NO. 41) |
| for ORF-2: | |
| 5′-UCCAGCAGCACAUCAAAAAG-3′; | (SEQ ID NO. 42) |
| 5′-CCAGUUUUUGCCCAUUCAGU-3′; | (SEQ ID NO. 43) |
| and | |
| 5′-UGACAAACCCACAGCCAAUA-3′. | (SEQ ID NO. 44) |

Thus, it is particularly preferred that the RNAi comprises at least a fragment, preferably at least 10 consecutive, more preferably 15, and most preferably 20 consecutive nucleotides from any one of SEQ ID NOS. 39-44. As discussed above, shorter stretches of said sequences, interspersed with features, such as hairpin loops, are also preferred.

For L1_{RP}, a CDS for ORF1 exists at position 907 to 1923 of SEQ ID NO. 27, encoding a protein sequence, given in SEQ NO. 45, and a CDS exists at 1987 to 5814 of SEQ ID NO. 27, encoding ORF2, the protein sequence being given in SEQ ID NO. 46. It is the protein encoded by ORF2 that is thought to have RT activity. Thus, it is preferred that the RNAi is directed to DNA comprised within positions 907 to 1923 of SEQ ID NO. 27 and/or 1987 to 5814 of SEQ ID NO. 27, and preferably capable of inhibiting expression of the proteins according to SEQ ID NO. 45 and, most preferably, SEQ ID NO. 46. Similarly, CDSs exist at positions 17717 to 18697 and 115033 to 116161 of SEQ ID NO. 32, but these are described as pseudogenes and are therefore, not preferred.

Thus, it is preferred that the RNAi comprises a stretch of RNA that corresponds to an RNA sequence encoding the proteins according to SEQ ID NO. 45 and, most preferably, SEQ ID NO. 46. The stretch may include hairpins or other features, discussed elsewhere, within it. Preferably, the RNAi consists of a 20 or 21 bp stretch of RNA that corresponds to an RNA sequence encoding the proteins according to SEQ ID NO. 45 and, most preferably, SEQ ID NO. 46. Preferably, the RNAi has sequence of SEQ ID NO. 19, or its RNA equivalent, SEQ ID NO. 47, which targets a consensus sequence in hot L1s.

In a further aspect, the invention provides such RNAi.

As described in Brouha (2003) et al, active L1s are preferably polymorphic, and preferably ‘young’ or recently formed, as the age of an L1 element or sequence determines its likely diversions. As discussed in Brouha (2003) et al, L1s with little sequence diversion were generally polymorphic in the population and were active in cultured cells. Conversely, highly diverged L1 sequences were most frequently fixed and inactive.

An active L1 is often 6 kbp in length, indicating that there is no 5′ truncation. Thus, the LINE-1 elements are preferably at least 6 kbp in length.

Preferred are the 80-100 retrotransposition-competent L1s predicted to be active in Brouha (2003) et al, in an average human being. Six of these retrotransposition-competent L1s were found to be ‘highly active L1s (hot L1s).’ Hot L1s preferably show at least ⅓ of the activity of L1_{RP}. Thus, it is particularly preferred that the L1 sequences are ‘hot L1s’, having a high biological activity in humans, and preferably show at least ⅓ of the activity of L1_{RP}.

L1_{RP} is a hot L1 and is described in Brouha et al (2003) and Hum. Mol. Genet. 8 (8), 1557-1560 (1999), available at http://hmg.oxfordjournals.org/cgi/reprint/8/8/1557, NCBI accession number AF148856, SEQ ID NO. 27. As it is preferred that the ORF's are targeted, nucleotide positions 907-1923 (ORF1) and 1987-5814 (ORF2) of SEQ ID NO. 27 are particularly preferred targets for the RNAi.

The activity relative to L1_{RP} may be measured by a suitable assay, for instance by linking the LINE-1 element to a detectable marker for expression, readily selected by the skilled person. Preferably, this may include the method used in Brouha et al (2003), an EGFP assay. The construction of an EGFP cassette and how to use this to assess activity is further described in reference number 23 from Brouha et al (2003), Haig H. Kazazian Jr et al: Nucleic Acids Research, 2000, Vol. 28, No. 6, 1418-1423. A suitable example of an L1 comprising EGFP is given in SEQ ID NO. 26, discussed below.

Without being bound by theory, it is thought that transcription is initiated from an internal promoter located within its 5 prime UTR, and the RNA is transported to the cytoplasm. The L1-encoded proteins, ORF1p and ORF2p, then act on the mRNA that encoded them, a phenomenon known as cis preference.

The resultant ribonucleoprotein particle then re-enterers the nucleus where L1 integration is thought to occur by target-primed reverse transcription. During this process, the L1 endonuclease generates a single-stranded nick in genomic DNA at the loose consensus sequence 5′-TTTTT/A-3′, exposing a 3′ OH, which is used as a primer for reverse transcription of L1 RNA by the L1 RT (reverse transcriptase).

Preferably, the LINE-1 sequence comprises the 21 base pair consensus sequence, SEQ ID NO. 19, its corresponding (antisense) DNA sequence or RNA equivalents thereof. Therefore, it is also preferred that the RNAi of the present invention comprises the RNA equivalent of this 21 base pair sequence, or the RNA equivalent of the corresponding DNA sequence to SEQ ID NO. 19, or a sequence that is capable of hybridising to it under stringent conditions, for instance 6×SSC.

It is also preferred that the sequence targeted by the RNAi is any of SEQ ID NO. 35-38, more preferably SEQ ID NO. 37 and most preferably SEQ ID NO. 35. Included in this is the corresponding DNA sequence or RNA equivalents thereof. SEQ ID NO. 35 is the 60 bp sequence targeted in Example 2, whilst SEQ ID NOS. 36-38 are longer consensus sequences, as discussed below.

The L1 sequence that is to be targeted can also share a degree of homology with any of the sequences described herein, preferably at least 70%, more preferably at least 85%, more preferably at least 95%, more preferably at least 99%, more preferably at least 99.5%, more preferably at least 99.9%, more preferably at least 99.95%, and most preferably at least 99.99%, homology. Thus, the RNAi preferably targets L1 elements comprising these preferred sequences or their corresponding sequences, or homologues thereof

Where reference is made to a particular sequence, it will be understood that this includes, where the sequence is a DNA sequence, its corresponding DNA sequence (for instance a sequence that hybridises to the aforementioned sequence, particularly under highly stringent conditions, such as 6×SSC) or RNA equivalents thereof, i.e. RNA sequences obtainable by transcription of said DNA sequences.

The LINE-1 elements (L1s) may be selected from a broad group of retrotransposable elements, providing that the element encodes RT. It is preferred that the L1s are derived from the ‘transcribed group A’ (Ta) subset of L1 elements or from the pre-Ta subset. However, the Ta subset is particularly preferred, especially the Ta-1d family.

However, it is especially preferred that the L1s are selected from the group consisting of LRE3, L1RP (NCBI accession number AF148856), and accession numbers ac004200, ac002980, al356438, al512428, ac021017, and al137845, SEQ ID NOS. 26-33, respectively. These sequences and their associated feature data are available from the NCBI website: http://www.ncbl.nim.nih.gov/entrez/query.fcgi.

With regard to SEQ ID NO. 26, the full sequence given is a synthetic construct of LRE3-EGFP, i.e. LRE3 tagged with EGFP (Enhanced Green Fluorescent Protein), together with a disrupted Dlgh2 gene (partial sequence). However, the skilled person will readily understand that it is not necessary to include the EGFP coding region at positions 1-155 nor the Dlgh2 gene portion at positions 510 to 910, and these may be replaced with ORF1 and ORF2, as present in the other L1s provided. This and further feature information is available from the NCBI website, under the accession number for SEQ ID NO 26, AY995186.

The skilled person will be able to engineer a L1 to include a detectable marker, such as EGFP, and may use SEQ ID NO. 26 as a starting point or template.

The consensus sequences for the groups of L1 elements are provided in SEQ ID NOS 36-38. SEQ ID NO 36 is the Ta-1d consensus sequence, SEQ ID NO 37 is the Hot element consensus sequence, and SEQ ID NO 38 is a broader consensus sequence for 90 active L1s. These were obtained from the ‘supporting information’ of the Brouha et al (2003) article online, available from www.pnas.org.

Therefore, it is preferred that the L1 sequence is selected from any of SEQ ID NOS. 36, 37 and 38 or its corresponding sequence or homologue. The homologue preferably shares at least 70%, more preferably at least 85%, more preferably at least 95%, more preferably at least 99%, more preferably at least 99.5%, more preferably at least 99.95%, and most preferably at least 99.99%, homology with said SEQ ID NO or its corresponding sequence.

The hot L1 consensus sequence, SEQ ID NO. 37, or its corresponding sequence or homologue is particularly preferred, due to their increased activity. The degree of homology or similarity to the hot 11 consensus is a good predictor of retrotransposition activity. Brouha et al (2003) analysed the relationship between L1 activity and nucleotide sequence, and constructed a consensus sequence (SEQ ID NO. 37 with eight of the hot L1s (LRE3, L1_{RP}, ac004200, ac002980, al356438, al512428, ac021017, and al137845). This sequence is identical to the Ta-1d consensus (SEQ ID NO. 36) except for a silent ORF1 change at position 1033 and is identical to a consensus of the 90 intact L1s (SEQ ID NO. 38), except for 12 polymorphic sites.

Brouha et al (2003) compared the L1s, first as active and inactive groups, then pairwise to the consensus of the hot elements. They analyzed the L1s in their entirety, then by region, and finally by whether differences resulted in amino acid changes. It was found that there were no nucleotide changes uniquely associated with active or inactive L1s. As expected, with some exceptions, the closer an L1 was to the hot L1 consensus, the more likely it was to be active. Taken with the above result, their data indicates that a decrease in retrotransposition activity occurs as a function of time. The ether an L1 is from the “hot” consensus sequence, the less likely it is to be active.

It is particularly preferred that the LINE-1 elements comprise a very high degree of sequence homology to the sequences given above, especially the hot L1s. The reason for this is that, as discussed in Brouha et al, the closer an L1 sequence was to a hot L1 sequence, the more likely it was to be active, although there are some exceptions. As mentioned above, it is preferred that the LINE-1 sequence or element is a hot L1 and preferably has at least ⅓ and preferably at least ⅔, more preferably 100% and most preferably greater than 100%, preferably 150% or more of the activity of L1_{RP}.

Of the especially preferred hot L1s, all are polymorphic and 3 (ac002980, ac004200, and al356438) come from the youngest Ta-1d group. In addition, one is in the Ta-1nd group (al512428), and another is a member of the younger Ta-0 sub groups (al137845). The sequences of these 5 canonical hot L1s are very similar to the consensus sequences of their respective groups or sub groups, indicating that they have retrotransposed relatively recently in human evolution. Finally, one hot L1 (ac021017) is non-canonical.

The 21 nucleotide sequence, SEQ ID No. 19, is complimentary to a corresponding sequence present within the 90 or so (around 80 to 100) active L1 retroelements, that are preferred targets of the present invention. Thus, the RNAi preferably targets L1 elements comprising this sequence or its corresponding sequence, or homologues thereof.

Whilst the link between LINE-1 and Reverse Transcriptase (RT) has been known for about thirty years, when it was made clear that RT is one of the genes encoded by LINE-1, the LINE-1 retroelements have traditionally been classified as useless and is often referred to as “junk DNA”. As such, this junk DNA was widely regarded as having no biological role. Despite this, the present inventors have surprisingly discovered that RT inhibition can antagonise tumour growth. The present inventors were the first to recognise that RNAi specifically targeted to LINE-1 sequences inhibits cell proliferation in culture and tumour growth in animal models.

Oncogene 2003, Vol. 22, PP 2750 to 2761, by Mangiacasale et al, focuses on the pharmacological RT inhibitors and makes no mention of RNAi at all. Cytogenet. Genome Res 2004, Vol. 105, PP 346 to 360 by C. Spadafora, one of the present inventors, is a review article bringing together various strands of the prior art, but does again not disclose the RNAi approach.

The RNAi is preferably double-stranded. Preferably, the double-stranded ribo-oligonucleotide is not used in the free form for cell transfection, but is preferably carried by a DNA construct encoding the specific siRNA. Preferably, the transcribed RNA forms a double-stranded palindromic structure that is further spontaneously processed by the cell “dicer” system, thus forming the siRNA molecules, for instance as taught in Brummelkamp et al, 2002. (NOTE to DMI insert this reference into the end of the reference lists.)

In an alternative aspect, it is preferred that the standard cell transfection procedure for delivery of RNAi is replaced by an appropriate delivery system of retroviral or adenoviral origin. Such viral systems are well known in the art. Preferably, the viral vector or capsid expressing the L1-targeted RNAi, preferably, siRNA, is able to specifically target tumour cells and, by infecting the tumour cells, lead to expression of the RNAi, for instance by transcription to provide the siRNA, thereby leading to the antagonism of tumour growth and the stimulation of differentiation of the tumour cells.

Although targeting or treatment of any tumour cell is envisaged, the tumour cell can preferably be selected from the group consisting of breast cancer tumours, lung cancer tumours, melanomas and prostate carcinomas. Indeed, melanomas and prostate carcinomas are particularly preferred.

Thus, it is particularly preferred that the RNAI is delivered direct to the cancerous tissue, for instance by injection or released from an implant, where appropriate. As mentioned above, it is particularly preferred that viral vectors or capsids are used, preferably comprising a DNA construct encoding the specific siRNA. In this instance, it is preferred that the viral vector or capsid is capable of expressing the DNA construct in the target tissue, i.e. the cancerous or tumour tissue. This may be by means of an appropriate tissue-specific promoter that is included in the DNA construct, and/or by means of viral capsids or vectors specific for particular tissues.

The above also applies to a plasmid comprising polynucleotides, preferably DNA, encoding the specific siRNA. In particular, it is preferred that this plasmid comprises the suitable tissue-specific promoter or other means for tissue-specific expression of the siRNA.

In both the instances of plasmids and vectors or capsids, it is preferred that the plasmid, vector or capsid targets only cancerous tissue, again preferably in a tissue-specific manner.

In a further aspect, the present invention also provides a method of treating a patient with cancer or a tumour, the method preferably comprising methods of RNA interference, as described above. Preferably, the method comprises selecting an individual with a cancerous tissue, and using methods of RNA interference against at least one L1 element, as discussed above. Preferably, the method includes a therapeutically effective inhibition of Reversed Transcriptase (RT), sufficient to lead to inhibition or blocking of proliferation of cancerous tissue and, preferably, to stimulate differentiation of said tissue.

Whilst it is envisaged that only one line 1 element is targeted, it is preferred that multiple line 1 elements are targeted, either at the same time or sequentially, as part of a planned regimen.

In a further aspect, the present invention also provides the use of a polynucleotide sequence, preferably a DNA sequence, encoding siRNA capable of targeting or recognising a portion of at least one LINE-1 element, for use in the manufacture of a medicament for the treatment of a cancerous condition. Preferably, the medicament comprises a viral capsid or vector, which in turn comprises the polynucleotide.

The present invention will now be illustrated further with respect to the accompanying, non-limiting examples.

### EXAMPLE 1

### Methods

### Cell Cultures.

Human A-375 melanoma (ATCC-CRL-1619), TVM-A12 primary melanoma-derived (20), HT29 adenocarcinoma (ATCC HTB-38), H69 small cell lung carcinoma (SCLC) (ATCC HTB119), and PC3 prostate carcinoma (ATCC CRL-1435) cell lines were seeded in six-well plates at a density of 10⁴ to 5×10⁴ cells/well and cultured in DMEM or RPMI1640 medium with 10% fetal bovine serum. Nevirapine and Efavirenz were purified from commercially available Viramune (Boehringer-Ingelheim) and Sustiva (Bristol-Myers Squibb) as described (18). The drugs were made 350 and 15 μM in dimethyl sulfoxide (DMSO, Sigma-Aldrich), respectively, and added to cells 5 h after seeding; the same DMSO volume (0.2% final concentration) was added to controls. Fresh RT inhibitor-containing medium was changed every 48 h. Cells were harvested every 96 h, counted in a Burker chamber (two countings/sample) and replated at the same density.

### Cell Cycle and Cell Death Analysis.

BrdU (20 μM) was added to the cultures during the last 30 min before harvesting. Harvested cells were then treated with anti-BrdU antibody and propidium iodide (PI) and subjected to biparametric analysis of the DNA content and BrdU incorporation in a FACStar Plus flow-cytometer (Beckton-Dickinson). Cell death was assessed by microscopy after combined staining with DAPI (nuclear morphology); PI (cell permeability); and 3,3 dihexyl-oxacarbocyanine [DiOC6(3)], a fluorescent probe for mitochondrial transmembrane potential.

### Indirect Immunofluorescence and Confocal Laser Scanning Microscopy.

A-375 and TVM-A12 cells were fixed with 4% para-formaldehyde for 10 min and permeabilized in 0.2% Triton-X 100 in PBS for 5 min. Mouse monoclonal anti-bovine α-tubulin (Molecular Probes, A-11126), was revealed by Alexa Fluor 488-conjugated secondary antibody (Molecular Probes, cat. A-11001); nuclei were stained with 2 μg/ml PI in the presence of 0.1 mg/ml ribonuclease A. Samples were imaged under a confocal Leica TCS 4D microscope equipped with an argon/krypton laser (excitation and emission wavelengths: 488 nm and 510 nm for Alexa 488, and 568 nm and 590 nm for PI). Confocal sections were taken at 0.5-1 μm intervals.

### Scanning Electron Microscopy (SEM).

A-375 and TVM-A12 cells were fixed in 2.5% glutaraldehyde in 0.1 M Millonig's phosphate buffer. After washing, cells were post-fixed with 1% OsO₄ (1 h, 4° C.) in MPB and dehydrated using increasing acetone concentrations. Samples were critical-point dried using liquid CO₂ and sputter-coated with gold before examination on a Stereoscan 240 scanning electron microscope (Cambridge Instr., Cambridge, UK).

### Semiquantitative RT-PCR.

RNA extraction and treatment with RNase-free DNase I were as described (18). cDNAs were synthesized using 300 ng of RNA, oligo (dT) and the Thermoscript system (Invitrogen). 1/25 of reaction mixtures was amplified using the Platinum Taq DNA Polymerase kit (Invitrogen) and 30 pmol of oligonucleotides (MWG-Biotech, Ebersberg, Germany) in an initial step of 2 min at 94° C., followed by cycles of 30 s at 94° C., 30 s at 58-62° C., 1 min at 72° C.

Each oligo pair was used in sequential amplification series with increasing numbers (25 to 40) of cycles. PCR products were electrophoresed, transferred to membranes and hybridized for 16 h at 42 C.° with [³²P]γ-ATP end-labelled internal oligonucleotides. The intensity of the amplification signal was measured by densitometry in at least three independent experiments for each gene.

### Oligonucleotides Used for Semi-Quantitative PCR Analysis Forward, F; Reverse, R) and Internal Probes (INT) for Hybridization

C-myc PCR product size, 633 bp;

| | | |
|---|---|---|
| F, | 5′-gtcacacccttctcccttcg-3′; | (SEQ ID NO. 1) |
| R, | 5′-tgtgctgatgtgtggagacg-3′; | (SEQ ID NO. 2) |
| INT, | 5′-agagaagctggcctcctacc-3′. | (SEQ ID NO. 3). |

Bcl2 PCR product size, 459 bp;

| | | |
|---|---|---|
| F, | 5′-ggtgccacctgtggtccacctg-3′; | (SEQ ID NO. 4) |
| R, | 5′-cttcacttgtggcccagatagg-3′; | (SEQ ID NO. 5) |
| INT, | 5′-ctgaagagctcctccaccac-3′. | (SEQ ID NO. 6) |

E-cadherin PCR product size, 732 bp;

| | | |
|---|---|---|
| F, | 5′-ctcctctcctggcctcagaa-3′; | (SEQ ID NO. 7) |
| R, | 5′-tactgctgcttggcctcaaa-3′; | (SEQ ID NO. 8) |
| INT | 5′-gaacgcattgccacatacac-3′. | (SEQ ID NO. 9) |

PSA PCR product size, 584 bp;

| | | |
|---|---|---|
| F, | 5′-ttgtcttcctcaccctgtcc-3′; | (SEQ ID NO. 10) |
| R, | 5′-agcacacagcatgaacttgg-3′; | (SEQ ID NO. 11) |
| INT, | 5′-ccacacccgctctacgatat-3′. | (SEQ ID NO. 12) |

Ccnd1 PCR product size, 690 bp;

| | | |
|---|---|---|
| F, | 5′-ccctcggtgtcctacttcaa-3′; | (SEQ ID NO. 13) |
| R, | 5′-tcctcctcttcctcctcctc-3′; | (SEQ ID NO. 14) |
| INT | 5′-cgcacgatttcattgaacac-3′. | (SEQ ID NO. 15) |

Gapdh PCR product size, 590 bp;

| | | |
|---|---|---|
| F, | 5′-aggggtctacatggcaactg-3′; | (SEQ ID NO. 16) |
| R, | 5′-acccagaagactgtggatgg-3′; | (SEQ ID NO. 17) |
| INT, | 5′-gtcagtggtggacctgacct-3′. | (SEQ ID NO. 18) |

### RNA Interference to LINE-1.

A 21-nt double-stranded siRNA oligonucleotide (L1-I) (5′-AAGAGCAACTCCAAGACACAT-3′, SEQ ID NO. 19) was designed to target the consensus sequence of the highly active LINE-1 elements described by Bruha et al. (21). Specifically, the following sequences were targeted:
i) eight hot L1s (LRE3 (SEQ ID NO. 26), L1RP (SEQ ID NO. 27), ac004200 (SEQ ID NO. 28), ac002980 (SEQ ID NO. 29), al356438 (SEQ ID NO. 30), al512428 (SEQ ID NO. 31), ac021017 (SEQ ID NO. 32), al1378459 (SEQ ID NO. 33));
ii) the Ta-1d family; and
iii) 90 full length L1 elements. For control, cells were transfected with a non-specific siRNA (SEQ ID NO 34) that was 3′-fluorescein modified to monitor the transfection efficiency.

siRNA oligonucleotides were synthesized by QIAGEN USA. Transfections were performed in A-375 cells using RNAiFect Transfection Reagent (QIAGEN cat. 301605) in 24-well plates adding 1, 5 μg siRNA per well. Cells were counted 48 and 72 h after transfection, and cell morphology was recorded under an Olympus CK30 inverted microscope equipped with an Olympus CAMEDIA digital camera. About 80% of cells were transfected after 24 h, as determined by fluorescence microscopy.

LINE-1 expression was analyzed by RT-PCR 48 h after transfection using specific pairs of primers for LINE-1 ORF-1 and ORF-2:

| | |
|---|---|
| ORF-1: | |
| F, 5′-AGAAATGAGCAAAGCCTCCA-3′; | (SEQ ID NO. 20) |
| R, 5′-GCCTGGTGGTGACAAAATCT-3′ | (SEQ ID NO. 21) |
| ORF-2: | |
| F, 5′-TCCAGCAGCACATCAAAAAG-3′; | (SEQ ID NO. 22) |
| R 5′-CCAGTTTTTGCCCATTCAGT-3′. | (SEQ ID NO. 23) |

RNA extraction and RT-PCR conditions were as described herein, except that the annealing T° C. was 54° C. and amplification was carried out through 23 cycles. Internal oligonucleotides for Southern analysis were: 5′-TAAGGGCAGCCAGAGAGAAA-3′ (ORF-1, SEQ ID NO. 24) and 5′-TGACAAACCCACAGCCAATA-3′ (ORF-2, SEQ ID NO. 25).

### Tumor Xenografts and Treatment of Animals.

Five-week old athymic nude mice (Harlan, Italy), kept in accordance with the European Union guidelines, were inoculated sub-cutaneously with A-375 melanoma (4×10⁶), H-69 (10⁷), PC3 (2×10⁶) and HT-29 (10⁶) cells in 100 μl PBS. Mice were sub-cutaneously injected daily five days a week with Efavirenz (20 mg/kg) using a 4 mg/ml stock in DMSO freshly diluted 1:1 with physiological solution. Controls were injected with 50% DMSO. Treatment started one day or one week after tumor implant, and, in some experiments, was discontinued after 14 days. Tumor growth was monitored every other day by caliper measurements; volumes were calculated using the formula:

length×width×height×0.52 (22).

### Results

### RNA Interference (RNAi) Targeted Against RT-Encoding LINE-1 Families Reduces Proliferation and Promotes Differentiation in Melanoma Cells

We wanted to ascertain unambiguously whether the reduced growth rate and induction of differentiation observed in response to pharmaceutical RT inhibitors, as discussed below, are actually attributable to the specific inhibition of cellular RTs. To address this, RNAi experiments were designed to specifically target LINE-1 elements subfamilies that are known to be most abundantly expressed in human cells (21, and the targeted sequences described in the corresponding section of the Methods, above).

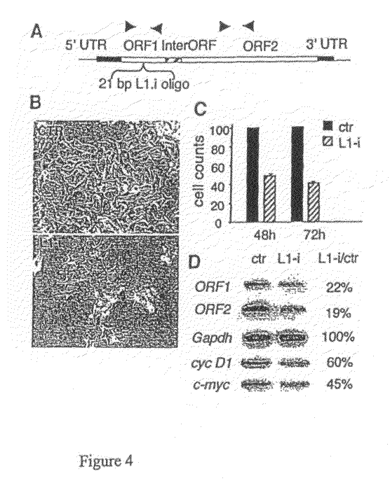

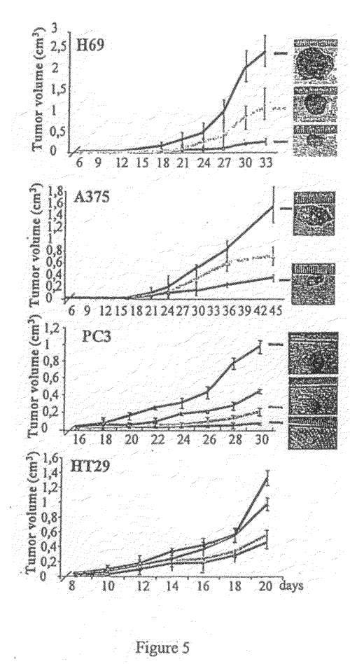

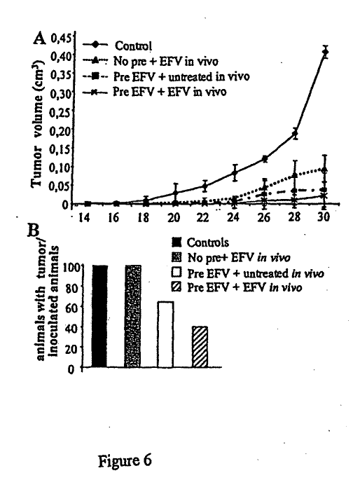

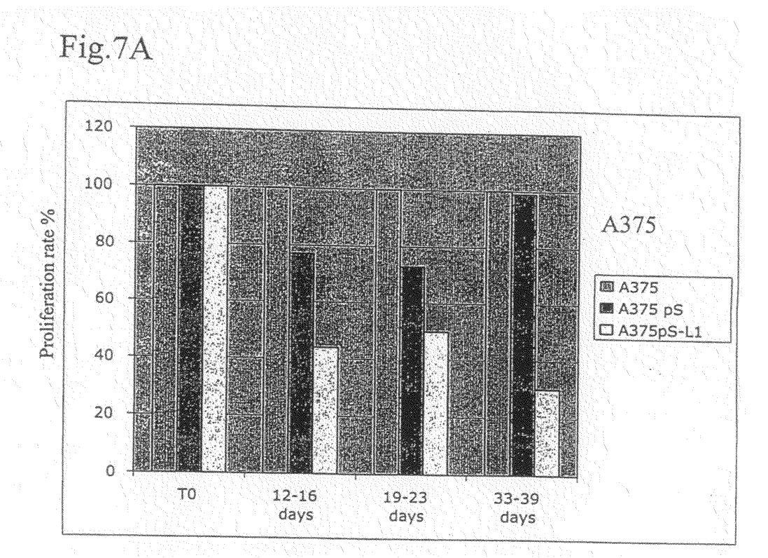

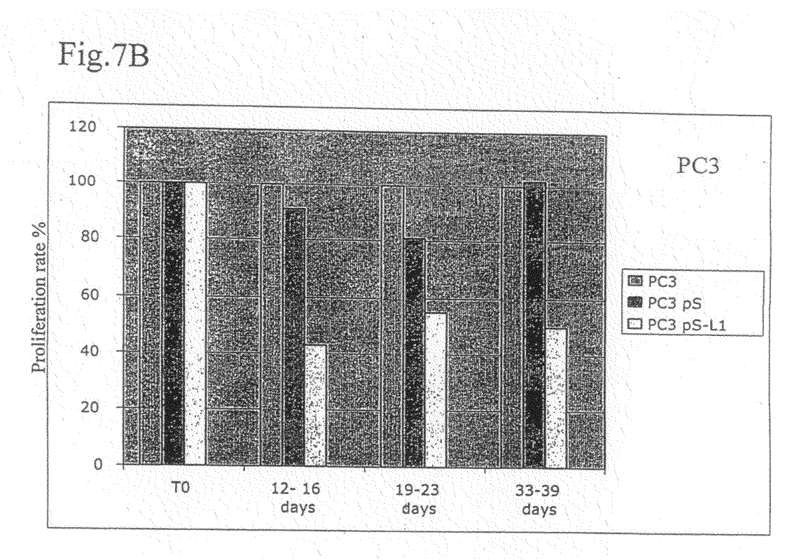

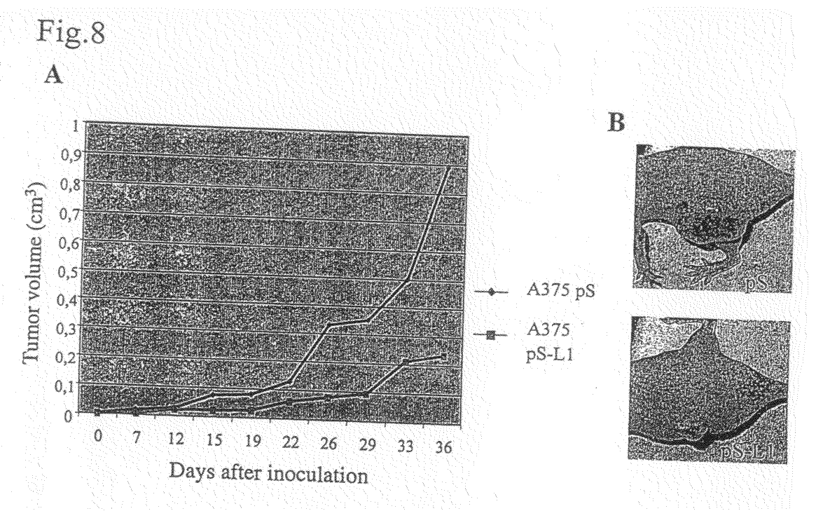

A double-stranded RNA oligonucleotide homologous to LINE-1 ORF1 (FIG. 4, panel A) was transfected in A-375 cells. 48-72 hours after transfection, a typical differentiated morphology (panel B) was induced. Concomitantly, proliferation decreased by about 70% (panel C) compared to cells transfected with non-specific oligonucleotide. These results are comparable to those obtained with pharmacological RT inhibition. By semi-quantitative RT-PCR analysis, expression of both ORF1 and ORF2 was reduced by almost 80% compared to cells transfected with non-specific oligonucleotide (panel D). Furthermore, RNAi to LINE-1 elements induced down-regulation of expression of the c-myc and cyclin-D1 genes, but not of GAPDH, as seen in response to RT inhibitory drugs.

### RT Inhibitors Reversibly Reduce Cell Proliferation

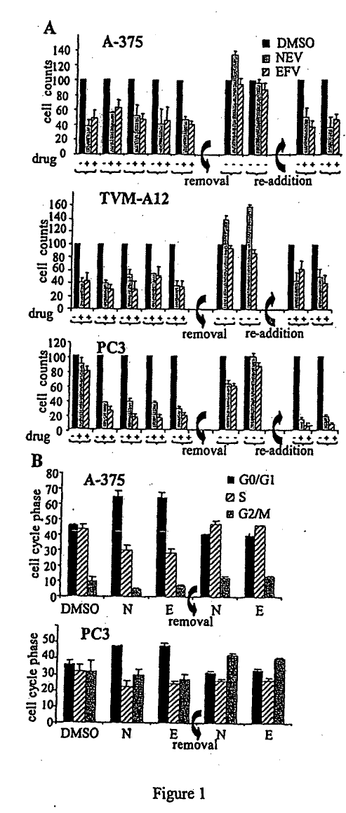

We also investigated the response of human transformed cell lines to prolonged exposure to two widely used RT inhibitors, i.e. nevirapine and efavirenz. Cultures from A-375 melanoma, PC3 prostate carcinoma and TVM-A12 primary melanoma-derived cell lines were passaged, counted and replated every 96 h with continuous drug re-addition for at least 20 days (five 96 h-cycles). As shown in FIG. 1A, both inhibitors effectively reduce cell growth in all cell lines, with a stable inhibitory effect during prolonged exposure. Growth inhibition was reversible: when RT inhibitors were removed, all cell lines resumed proliferation at a comparable rate to controls within one or two 96 h-cycles. Re-addition of the drugs inhibited again proliferation in all cell lines. Thus, the reduction of cell growth associated with RT inhibition is not inherited as a permanent change through cell division.

To elucidate the basis of reduced proliferation, we investigated whether either RT inhibitor induced cell death in A-375 or PC3 cell lines. Combined staining with PI to reveal permeable necrotic cells, DAPI to visualize apoptotic nuclei, and DiOC6(3) to monitor the loss of mitochondrial transmembrane potential, revealed no significant induction of cell death by either RT inhibitor; what low ratio was recorded (15% at most after 72 h of exposure to either drug) was largely accounted for by apoptosis (data not shown). Thus, neither drug has significant non-specific toxicity, suggesting that reduced cell growth rather reflects the induction of cell cycle delay.

To assess this, we employed biparametric FACS analysis to measure the DNA content (revealed by PI) and DNA replication (by BrdU incorporation) after four 96 h-cycles of exposure to RT inhibitors. The cell cycle profile was significantly altered in anti-RT treated cultures, showing an increased proportion of BrdU-negative cells with a G0/G1 content that was especially pronounced in A-375 cell cultures (FIG. 1B). Removal of the drugs re-established the original cell cycle profile and abolished the G1 delay.

### Nevirapine Induces Morphological Differentiation, and Expression of Differentiation and Proliferation Genes, in Transformed Cell Lines

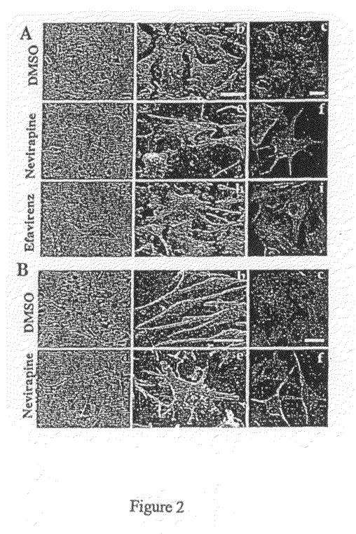

Since melanomas are resistant to most therapeutic treatments, it was relevant to determine whether RT inhibitors induced differentiation concomitant with reduced cell growth. We first examined A-375 melanoma cells, which can acquire a typical dendritic-like phenotype in response to certain inducers of differentiation (23). As shown in FIG. 2A, morphological differentiation, revealed by cell shape, dendritic-like extensions and increased adhesion, became evident within four-five days of exposure to nevirapine (d) or efavirenz (g), compared to DMSO-treated controls (a). By scanning electron microscopy (SEM), A-375 cells cultured with nevirapine (e) and efavirenz (h) become flattened compared to untreated controls (b) and exhibit elongated dendritic extensions that adhere tightly to the substrate. Confocal microscopy after α-tubulin immunostaining further revealed that microtubule arrays are reorganized throughout the length of outgrowing dendrites in RT-inhibited cells (f-i), different from controls (c), in which short microtubules concentrate around the nucleating centers.

A similar response was observed in primary TVM-A12 cells derived from melanoma after nevirapine treatment (FIG. 2B): untreated cells have a spindle-shaped morphology by phase contrast (a) and SEM (b); nevirapine-treated TVM-A12 cells formed instead typical branched dendrites (d-e) and displayed well-organized, elongated microtubule arrays (f), compared to untreated cells (c).

The induction of morphological differentiation suggests that critical regulatory genes are modulated in response to the RT inhibitory treatment. This was investigated in semiquantitative RT-PCR analysis of cultures treated with DMSO only, or nevirapine or efavirenz for four cycles. In A-375 melanoma cells, we focussed on a set of four genes: the E-cadherin gene, involved in cell-cell adhesion and expressed in differentiated but not in tumor cells (24); and the c-myc, bcl-2 (25) and cyclin D1 (26) genes, which are directly implicated in melanoma cell proliferation and tumor growth.

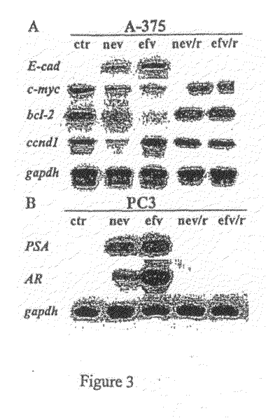

As shown in FIG. 3A, we found the E-cadherin gene is markedly up-regulated in RT-inhibited A-375 cultures compared to controls; in contrast, c-myc, bcl-2 and cyclin D1 genes are down-regulated. One exception was recorded for efavirenz, which failed to down-regulate cyclin D1 expression. We also analysed PC3 prostate carcinoma cells and selected two marker genes of the differentiated prostatic epithelia, i.e. the prostate-specific antigen PSA (27) and androgen receptor (AR) (28) genes. Neither of these genes is expressed in untreated cultures, yet both genes were induced in response to RT inhibitors (FIG. 3B). Again, the expression of all genes returned to the original level when the inhibitors were removed. Thus, RT inhibitory drugs yield the reprogramming of expression of critical genes in transformed cells, consistent with the induction of differentiation, yet this reprogramming is reversible and is abolished when RT-inhibition is released.

### RT Inhibitors Reduce the Growth of Human Tumor Xenografts in Athymic Nude Mice

Since critical features of transformed cells, including proliferation and differentiation, are modulated by RT inhibition, we tested the ability of RT inhibitors to antagonize tumor growth in vivo.

Tumorigenic cell lines selected for these experiments include A-375 and PC3 lines, as well as HT29 colon and H69 small cell lung carcinoma lines, which also showed reduced cell growth in response to RT inhibitors (19, and data not shown). Cells were inoculated subcutaneously in the limb of athymic nude mice. Animals were then subjected to treatment with efavirenz, because this drug had shown a higher in vivo effectiveness than nevirapine in preliminary assays. The optimal dose (20 mg/kg body weight) was determined in dose-response experiments testing 4 to 40 mg/kg of the drug.

The efavirenz treatment proved safe in all animal groups, with no animal death or explicit signs of toxicity in any of the groups—though the group treated with 40 mg/kg showed a significant decrease of body weight in more than 60% of animals. FIG. 5 shows the recorded curves of tumor growth in mice untreated (red) or treated with efavirenz, starting one day (purple), or one week (yellow), after tumor inoculation.

Tumor growth was markedly reduced in treated compared to untreated animals for all xenograft types, and tumor progression was antagonized with comparable effectiveness regardless of the timing of the treatment start, despite of differences in the initial tumor size. The growth curves of PC3- and HT29-derived tumors in animals treated from day one after inoculation, but subjected to treatment discontinuation after day 15 (green curves), demonstrate that RT-dependent inhibition of tumor growth is reversible in vivo.

### Efavirenz-Treated PC3 Cells Exhibit Reduced Tumorigenicity In Vivo

We also investigated whether pretreatment of transformed cells with efavirenz would modify the tumorigenic potential of derived xenografts. PC3 prostate cancer cells were cultured with 20 μM efavirenz for two 96 h-cycles, a time that was sufficient for induction of the PSA and AR genes (FIG. 3B), and subsequently inoculated in nude mice.

Untreated cells were inoculated in parallel batches of animals. Efavirenz-pretreated, or untreated, PC3 cell xenografts were then either continuously treated with efavirenz in vivo or were left untreated. As shown in FIG. 6A, untreated PC3 cells develop aggressive tumors in all animals. In contrast, efavirenz-pretreated PC3 cells showed a reduced ability to form tumors in vivo and xenografts grew more slowly. As summarized in FIG. 6 B, efavirenz-pretreated PC3 cells developed slowly-growing xenografts in 65% of the inoculated animals, compared to 100% using untreated cells. Moreover, only 40% of the animals inoculated with pretreated cells and further treated with efavirenz in vivo developed a tumor at all, and in that case the growth curve was flat. Thus, efavirenz attenuates the tumorigenic potential of transformed cells.

### Discussion

This work highlight three features of the human genome that have implications for cancer: first, LINE-1 elements are identified as active components of a mechanism involved in control of cell differentiation and proliferation; second, RNAi-dependent inactivation of LINE-1 elements, or pharmacological inhibition of the endogenous RT activity which they encode, can restore control of these traits in transformed cells; third, inhibitors of RT reduce tumor growth in animal models in vivo.

The RT inhibitor drugs used in this work, nevirapine and efavirenz, share a common mechanism of action by binding the hydrophobic pocket in the p66 subunit of RT enzymes (29,30). Though being designed to target the HIV-encoded RT, both inhibit the enzymatic activity of the endogenous RT in non-infected cells in vitro (19). We have now shown that both drugs reduce proliferation of transformed cells, largely independent on cell death, but associated with G1 delay or arrest. Concomitant with this, RT inhibitors induce morphological differentiation of transformed cells. The induction of differentiation is rapid, different from the phenotypic changes elicited by inhibitors of the telomerase-associated RT (TERT), which require long treatment times (120 days) (31). Furthermore, we did not observe the reorganization of actin stress fibers or focal adhesion sites typical of senescent cells. The absence of senescence-specific modifications, and the rapid induction of differentiation, indicate that the RT inhibitors used here do not target TERT and induce a low-proliferating differentiated phenotype rather than senescence.

What was particularly surprising was that the specificity of RT inhibitory effects was demonstrated in RNAi experiments targeted against a subgroup of six LINE-1 retroposons that are highly expressed in human cells, accounting for 84% of the overall retrotransposition capability (21). Remarkably, we found that RNAi reduced expression of LINE-1-derived ORF1 and ORF2 by some 80% in A-375 cells, suggesting that the biologically active LINE-1 subgroup was efficiently down-regulated. Changes induced by RNAi to RT-encoding LINE-1 elements are indistinguishable from those caused by pharmacological RT inhibitors, implicating LINE-1 in control of cell proliferation and differentiation. The similarity of the phenotypes observed using independent approaches indicates that inhibition of LINE-1 expression, or of RT activity, is sufficient to delay proliferation and promote differentiation. These observations indicate that any unknown side effect of the drugs do not contribute to the observed phenotype in a non-specific manner.

Consistent with the induction of reduced growth and differentiated morphology, we found that expression of a panel of selected genes was reprogrammed in response to RT inhibition. This indicates that RT activity can effectively modulate the expression of genes that promote the transition from highly proliferating, transformed phenotypes to low proliferating, differentiated phenotypes, suggesting that genome function is the ultimate target of pharmaceutical or RNAi-dependent inhibition of RT activity. However, changes in gene expression are not inherited through cell division, but are reversible when RT inhibition is released. The reversibility of examined features, and their dependence on the presence of inhibitory drugs, is consistent with the notion that LINE-1-encoded RT is part of an epigenetic mechanism that modulates gene expression and has a role in the molecular mechanisms underlying cell proliferation and differentiation.

An aspect of this study is in the ability of RT inhibitory drugs to reduce tumor growth in nude mice inoculated with four human xenograft models in vivo. Tumor growth was inhibited as long as the animals were supplied with RT inhibitor, yet was resumed on discontinuation of the treatment, as observed in cell lines. While this data illustrates the promising cytostatic ability of RT inhibitors in cancer treatment, it confirms an epigenetic role of endogenous RTs in tumor growth. Furthermore, in vitro pretreatment of PC3 prostate carcinoma cells with efavirenz attenuates their tumorigenicity in vivo. Thus, the activation of differentiation markers and reduced proliferation associated with RT inhibition are part of a large-scale reprogramming that can attenuate the malignant phenotype of transformed cells in vivo.

Growing data indicate that epigenetic changes can reprogram tumor cells and convert the transformed phenotype into a ‘normal’ non-pathological state (32,33). Epigenetic reprogramming can bypass the genetic alterations that originally caused the malignant transformation in a variety of tumors (32). Therefore, epigenetic regulatory factors are viewed as valuable, worth-challenging targets in tumor therapy (34). However, many tested compounds have generally proven toxic and/or chemically unstable. Nevirapine and efavirenz have been used in AIDS treatment for many years: the prospect of using these RT inhibitors in cancer therapy would have obvious advantages given their epidemiological record of generally good tolerance to continued administration. Furthermore, epidemiological evidence indicate that Kaposi's sarcoma (35) and other AIDS-related cancers (36) have a reduced incidence in patients treated with highly active antiretroviral therapy (HAART): while this is generally viewed as a reflection of the improved immune reaction in treated patients, it may also suggest a direct inhibitory effect of HAART on the endogenous RT activity in tumor cells.

At this stage, the mechanism(s) through which RT activity can instruct the cell fate remain unclear. Retroposons can contribute to heterochromatin formation in fission yeast (37). Though such a mechanism has not been proved in higher eukaryotes, work in our laboratory suggest that LINE-1-encoded RT is implicated in the redistribution of DNA methylation and chromatin remodeling-dependent regulation of gene expression.

In synthesis, the endogenous RT emerges as a ‘functional’ marker of the cellular machinery associated with high proliferation and loss of differentiation, and can be regarded as a novel potential target in cancer therapy. Data may be particularly encouraging for prostate cancer, where the loss of AR expression is the main cause of hormone therapy failure. Since RT inhibitors up-regulate both the AR and PSA genes, these differentiation-inducing compounds might be useful to restore androgen sensitivity in prostate cancer cells.

### EXAMPLE 2

### A) Cloning of a LINE-1-Targeted siRNA Expressing DNA Construct

The siRNA-targeted sequence was derived from a LINE-1 element known to be highly active in human cells (Brouha et al., 2003, supplementary material). The targeted sequence was 60 bp-long (from 1492 to 1552), and is represented by SEQ ID NO. 35, and was artificially synthesized as a double stranded DNA. The DNA oligonucleotide was then cloned in the commercially available vector pSuper.retro.neo+GFP (OligoEngine, USA, cat.#VEC-PRT-0006)

### B) Assembling of a Retroviral Vector

The costruct was then transfected in retrovirus-producing Phi-NX cells (obtained from ATCC), was packaged in the newly synthesized retroviral particles and spontaneously released from the cells into the medium. 48 hours after transfection, retroviral particles (called pS-L1i) were collected by centrifugation, filtered through a 0.45 micrometre Millipore filter and used for cell infection. This protocol was carried out according to manufacturer's recommendation (OligoEngine).

### C) Infection of Human Tumorigenic Cell Lines

A375 (melanoma) and PC3 (prostate carcinoma) human cell lines were infected with pS-L1i by simply mixing the supernatant of transfected cells with cell cultures and incubating for 24 hours. Transfected cells were then selected in the presence of neomicyn for 7 days. All neo-resistant cells were found to be positive for the expression of the GFP reporter gene. As a control, parallel cultures from the same cell lines were infected with retroviral particles containing the empty DNA vector (pS) devoid of the siRNA-coding sequence.

As shown in FIG. 7, cells infected with pS-L1 exhibit a drastic reduction of proliferation, which remained constant for at least 39 days. Non-infected cells maintained a high proliferation rate, and pS-infected cells showed a moderate reduction of proliferation in the first few days after infection, but subsequently resumed quickly a high proliferation rate comparable to that of non-infected cells.

In good correlation with these data, the expression of LINE-1 (both ORF1 and ORF2) was strongly down-regulated in pS-L1, but not in pS-infected cells, both at the RNA and at the protein levels, as revealed by western blot analysis using a specific antibody (data not shown).

### D) pS-L1 Infected A375 Cells have a Reduced Tumorigenicity as Determined in in Vivo Assays by Inoculation in Nude Mice

To assess whether the tumorigenic potential of pS-L1i cells was affected, 5×10⁶ A375 cells infected with pS vector, or with LINE1-knock-out pS-L1i construct, were intradermally inoculated (15 days after infection) in two group of athymic nude mice. Tumor progression was then monitored in both groups of mice: FIG. 8, panel A, shows progression of tumor growth in mice inoculated with A375 pS and with A375 pS-L1i cells. The examples in panel B, show that tumor growth was markedly reduced in mice inoculated with LINE1-interfered cells as compared to those inoculated with control cells.

Together these results support the conclusions that LINE1 gene activity contributes to proliferation of transformed cells and can be regarded as novel potential target in gene cancer therapy.

Therefore, in order to improve the exploitation of the invention by gene therapy, the following two improvements have been introduced into the described method:
i) the double-stranded ribo oligonucleotide is not used in the free form for cell transfection, but is carried by a DNA construct encoding the specific siRNA. The transcribed RNA forms a double stranded palindromic structure that is further spontaneously processed by the cell “dicer” system thus forming the siRNA molecules (Brummelkamp et al., 2002, reference 38)
ii) the standard cell transfection procedure must be replaced by an appropriate delivery system of retroviral or adenoviral origin.

In other words, the improvement of the method consists in the development of a viral vector expressing LINE-1-targeted siRNA that will be used to infect the tumor. The LINE-1-targeted siRNA expression construct will be delivered in tumorigenic cells by the viral vector, thus inhibiting the expression of endogenous LINE-1. Based on our previous experience, constitutive functional knock-out of LINE-1 obtained in this manner will strongly antagonize tumor progression.

### REFERENCES

1. Deininger, P.-L., Moran, J.-V., Batzer, M.-A. & Kazazian, H.-H. (2003) Curr Op Gen Dev; 13, 651-8.
2. Kiessling, A.-A., Crowell, R. & Fox, C. Proc. (1989) Acad. Natl. Sci. USA 86,
3. Giordano, R., Magnano, A.-R., Zaccagnini, G., Pittoggi, C., Moscufo, N., Lorenzini, R. & Spadafora, C. (2000) J. Cell Biol. 148, 1107-1113
4. Poznanski, A.-A.& Calarco, P.-G. (1991) Dev Biol. 143, 271-81.
5. Packer, A.-I., Manova, K. & Bacharova, R.-F. (1993) Dev Biol. 157, 281-3.
6. Mwenda, J.-M. (1993) Cell Mol. Biol. 39, 317-28.
7. Deragon, J.-M., Sinnett, D. & Labuda, D. (1990) EMBO J. 9, 3363-8.
8. Martin, S.-L. (1991) Mol Cell Biol. 11, 4804-7.
9. Martin, S.-L. & Branciforte, D. (1993) Mol Cell Biol. 13, 5383-2.
10. Hagan, C.-R; Sheffield, R.-F. & Rudin, C.-M. (2003) Nat Genet. 35, 219-20.
11. Li, T.-H. & Schmid, C.-W. (2001) Gene. 276, 135-41.
12. Hagan, C.-R. & Rudin, C.-M. (2002) Am J Pharmacogenomics. 2, 25-36.
13. Khan, A.-S., Muller, J.& Sears, J.-F. (2001) Virus Res. 79, 39-45.
14. Friedlander, A.& Patarca, R. (1999) Crit Rev Oncogenesis. 10, 129-59
15. Ostertag, E.-M.& Kazazian, H.-H. Jr. (2001) Annu Rev Genet. 35, 501-38.
16. Kuo, K.-W., Sheu, H.-M., Huang, Y.-S.& Leung, W.-C. (1998) Biochem Biophys Res Comm. 253, 566-70.
17. Crone, T.-M., Schalles, S.-L., Benedict, C.-M., Pan, W., Ren, L., Loy, S.-E., Isom, H. & Clawson, G.-A. (1999) Hepathology. 29, 1114-23.
18. Pittoggi, C., Sciamanna, I., Mattei, E., Beraldi, R., Lobascio, A.-M., Mai, A., Quaglia, M.-G., Lorenzini, R. & Spadafora, C. (2003) Mol. Reprod. Dev. 66: 225-36.
19. Mangiacasale, R., Pittoggi, C., Sciamanna, I., Careddu, A., Mattei, E., Lorenzini, R., Travaglini, L., Landriscina, M., Barone, C., Nervi, C., Lavia, P. & Spadafora, C. (2003) Oncogene. 22, 2750-61.
20. Melino, G., Sinibaldi Vallebona, P., D'Atri, S., Annichiarico-Petruzzelli, M., Rasi, G., Catani, M. V., Tartaglia, M. I., Vemole, P., Spagnoli, L. G., Finazzi-Agrò, A. & Garaci, E. (1993) Clin Chem Enzym Comms. 6, 105-19.
21. Brouha, B., Schustak, J., Badge, R.-M., Lutz-Prigge, S., Farley, A.-H., Moran, J.-V. & Kazazian, H.-H., Jr. (2003) Proc Natl Acad Sci. USA 100, 5280-5285, available at: http://www.pnas.org/cgi/content/full/100/9/5280. Supporting information is available at: http://www.pnas.org/cgi/content/full/0831042100/DC1
22. Umekita, Y., Hiipakka, R.-A., Koknotis, J.-M. & Shutsung, L. (1996) Proc Natl Acad Sci. USA 93, 11802-7.
23. Sauane, M., Gopalkrishnan, R.-V., Sarkar, D., Su, Z.-Z., Lebedeva, I.-V., Dent, P., Pestka, S., & Fisher, P.-B. (2003) Cyt Growth Fac Rev. 14, 35-51.
24. Hsu, M.-Y., Meier, F.-E., Nesbit, M., Hsu, J.-Y., Van Belle, P., Elder, D.-E., & Herlyn, M. (2000) Am J Pathol. 156, 1515-25.
25. Utikal, J., Leiter, U., Udart, M., Kaskel, P., Peter, R.-U. & Krahn, G.-M. (2002) Cancer Invest. 20, 914-21.
26. Sauter, E.-R., Yeo, U.-C., Von Stemm, A., Zhu, W., Litwin, S., Tichansky, D.-S., Pistritto, G., Nesbit, M., Pinkel, D., Herl, M. & Bastian, B.-C. (2002) Cancer Res. 62, 3200-6.
27. Lilja, H. (2003) Urology. 62 (5, Suppl 1), 27-33.
28. Linja, M.-J., Savinainem, K.-J., Saramaki, O.-R., Tammela, T.-L., Vessella, R.-L. & Visakorpi, T. (2001) Cancer Res. 61, 3550-5.
29. Di Marzo Veronese, F., Copeland, T.-D., De Vico, A.-L., Rahman, R., Oroszlan, S., Gallo, R.-C. & Sarngadharan, M.-G. (1986) Science. 231, 1289-91.
30. Ren, J., Nichols, C., Bird, L., Chamberlain, P., Weaver, K, Short, S., Stuart, D.-I. & Stammers, D.-K. (2001) J Mol Biol; 312, 795-805.
31. Damm, K., Hemmann, U., Garin-Chesa, P., Hauel, N., Kauffmann, I., Priepke, H., Niestroj, C., Daiber, C., Enekel, B., Guilliard, B., Lauritsch, I., Muller, E., Pascolo, E., Sauter, G., Pantic, M., Martens, U. M., Wenz, C., Lingner, J., Kraut, N., Rettig, W.-J., & Schnapp, A. (2001) EMBO J. 20, 6958-68.
32. Lotem, J.& Sachs, L. (2002) Cancer Biol. 12, 339-46.
33. Li, L., Connelly, M.-C., Wetmore, C., Curran, T. & Morgan, J.-I. (2003) Cancer Res. 63, 2733-6.
34. Egger, G., Liang, G., Aparicio, A. & Jones, P. A. (2004) Nature. 429, 457-63.
35. Portsmouth, S., Stebbing, J., Gill, J., Mandalia, S., Bower, M., Nelson, M., Bower, M., & Gazzard, B. (2003) AIDS. 17, F17-F22.
36. Tirelli, U. & Bernardi, D. (2001) Eur J Cancer. 37, 1320-4.
37. Schramke, V. & Allshire, R. (2003) Science. 301, 1069-74.
38. Brummelkamp T R, Bernards R and Agami R (2002) a system for stable expression of short interfering RNAs in mammalian cells. Science 296, 550-552

Additional Information on L1_{RP}:

NCBI accession number AF148856, available at:
http://www.ncbi.nlm.nih.gov/entrez/viewer.fcgi?db=nucleotide&val=5070620, and described in:
REFERENCE 39 (bases 1 to 6019)
AUTHORS Schwahn, U., Lenzner, S., Dong, J., Feil, S., Hinzmann, B., van Duijnhoven, G., Kirschner, F, Hemberger, M., Bergen, A. A., Rosenberg, T., Pinckers, A. J., Fundele, R., Rosenthal, A., Cremers, F. P., Ropers, H. H. and Berger, W.
TITLE Positional cloning of the gene for X-linked retinitis pigmentosa 2
JOURNAL Nat. Genet. 19 (4), 327-332 (1998)
PUBMED 9697692
   REFERENCE 40 (bases 1 to 6019)
AUTHORS Kimberland, M. L., Divoky, V., Prchal, J., Schwahn, U., Berger, W. and Kazazian, H. H. Jr.
TITLE Full-length human L1 insertions retain the capacity for high frequency retrotransposition in cultured cells
JOURNAL Hum. Mol. Genet. 8 (8), 1557-1560 (1999)
PUBMED 10401005
   REFERENCE 41 (bases 1 to 6019)
AUTHORS Kimberland, M. L., Kazazian, H. H. and Schwahn, U.
TITLE Direct Submission
JOURNAL Submitted (6 May 1999) Genetics, School of Medicine, University of Pennsylvania, 515 CRB, 415 Curie Blvd., Philadelphia, Pa. 19104, USA

### Important Sequences

SEQ ID NO. 47:
5′-AAGAGCAACUCCAAGACACAU-3′
SEQ ID NO. 45:
MGKKQNKTGNSKTQSASPPPKERSSSPATEQSWMENDFDELREEGFRRSN
YSELREDIQTKGKEVENFEKNLEECITRITNTEKCLKELMELKTKARELR
EECRSLRSRCDQLEERVSAMEDEMNEMKREGKFREKRIKRNEQSLQELWD
YVKRPNLRLIGVPESDVENGTKLENTLQDIIQENFPNLARQANVQIQEIQ
RTPQRYSSRRATPRHIIVRFTKVEMKEKMLRAAREKGRVTLKGKPIRLTA
DLSAETLQARREWGPIFNILKEKNFQPRISYPAKLSFISEGEIKYFIDKQ
MLRDFVITR PALKELLKEALNMERNNRYQ PLQNHAKM 338
SEQ ID NO. 46:
MTGSTSHITILTLNINGLNSAIKRHRLASWIKSQDPSVCCIQETHLTCRD
THRLKIKGWRKIYQANGKQKKAGVAILVSDKTDFKPTKIKRDKEGHYIMV
KGSIQQEELTILNIYAPNTGAPRFIKQVLSDLQRDLDSHTLIMGDFNTPL
STLDRSTRQKVNKDTQELNSALHQADLIDIYRTLHPKSTEYTFFSAPHHT
YSKIDHIVGSKALLSKCKRTEIITNYLSDHSAIKLELRIKNLTQSRSTTW
KLNNLLLNDYWVHNEMKAEIKMFFETNENKDTTYQNLWDAFKAVCRGKFI
ALNAYKRKQERSKIDTLTSQLKELEKQEQTHSKASRRQEITKIRAELKEI
ETQKTLQKINESRSWFFERINKIDRPLARLIKKKREKNQIDTIKNDKGDI
TTDPTEIQTTIREYYKHLYANKLENLEEMDTFLDTYTLPRLNQEEVESLN
RPITGSEIVAIINSLPTKKSPGPDGFTAEFYQRYKEELVPFLLKLFQSIE
KEGILPNSFYEASIILIIPKPGRDTTKKENFRPISLMNIDAKILNKILAN
RIQQHIKKLIHHDQVGFIPGMQGWFNIRKSINVIQHINRAKDKNHMIISI
DAEKAFDKIQQPFMLKTLNKLGIDGTYFKIIRAIYDKPTANIILNGQKLE
AFPLKTGTRQGCPLSPLLFNIVLEVLARAIRQEKEIKGIQLGKEEVKLSL
FADDMIVYLENPIVSAQNLLKLISNFSKVSGYKINVQKSQAFLYTNNRQT
ESQIMGELPFTIASKRIKYLGIQLTRDVKDLFKENYKPLLKEIKEETNKW
KNIPCSWVGRINIVKMAILPKVIYRFNAIPIKLPMTFFTELEKTTLKFIW
NQKRARIAKSILSQKNKAGGITLPFKLYYKATVTKTAWYWYQNRDIDQWN
RTEPSEIMPHIYNYLIFDKPEKNKQWGKDSLFNKWCWENWLAICRKLKLD
PFLTPYTKINSRWIKDLNVKYKTIKTLEENLGITIQDIGVGKDFMSKTPK
AMATKDKIDKWDLIKLKSFCTAKETTIRVNRQPTTWEKIFATYSSDKGLI
SRIYNELKQIYKKKTNNPIKKWAKDMNRHFSKEDIYAAKKHMKKCSSSLA
IREMQIKTTMRYHLTPVRAMIIKKSGNNRCWRGCGEIGTLLHCEEDCKLV
QPLWKSVWRFLRDLELEIPFDPAIPLLGIYPNEYKSCCYKDTCTRMFIAA
LFTIAKTWNQPKCPTMIDWIKKMWHIYTMEYYAAIKNDEFISFVGTWMKL
ETIILSKLSQEQKTKHRIFSLIGGN 1275
SEQ ID NO 36
(a) Ta-1d consensus
6,021 nucleotides
5′-GGGGGAGGAGCCAAGATGGCCGAATAGGAACAGCTCCGGTCTACAGC
TCCCAGCGTGAGCGACGCAGAAGACGGTGATTTCTGCATTTCCATCTGAG
GTACCGGGTTCATCTCACTAGGGAGTGCCAGACAGTGGGCGCAGGCCAGT
GTGTGTGCGCACCGTGCGCGAGCCGAAGCAGGGCGAGGCATTGCCTCACC
TGGGAAGCGCAAGGGGTCAGGGAGTTCCCTTTCCGAGTCAAAGAAAGGGG
TGACGGACGCACCTGGAAAATCGGGTCACTCCCACCCGAATATTGCGCTT
TCAGACCGGCTTAAGAAACGGCGCACCACGAGACTATATCCCACACCTGG
CTCGGAGGGTCCTACGCCCACGGAATCTCGCTGATTGCTAGCACAGCAGT
CTGAGATCAAACTGCAAGGCGGCAACGAGGCTGGGGGAGGGGCGCCCGCC
ATTGCCCAGGCTTGCTTAGGTAAACAAAGCAGCCGGGAAGCTCGAACTGG
GTGGAGCCCACCACAGCTCAAGGAGGCCTGCCTGCCTCTGTAGGCTCCAC
CTCTGGGGGCAGGGCACAGACAAACAAAAAGACAGCAGTAACCTCTGCAG
ACTTAAGTGTCCCTGTCTGACAGCTTTGAAGAGAGCAGTGGTTCTCCCAG
CACGCAGCTGGAGATCTGAGAACGGGCAGACTGCCTCCTCAAGTGGGTCC
CTGACTCCTGACCCCCGAGCAGCCTAACTGGGAGGCACCCCCCAGCAGGG
GCACACTGACACCTCACACGGCAGGGTATTCCAACAGACCTGCAGCTGAG
GGTCCTGTCTGTTAGAAGGAAAACTAACAACCAGAAAGGACATCTACACC
GAAAACCCATCTGTACATCACCATCATCAAAGACCAAAAGTAGATAAAAC
CACAAAGATGGGGAAAAAACAGAACAGAAAAACTGGAAACTCTAAAACGC
AGAGCGCCTCTCCTCCTCCAAAGGAACGCAGTTCCTCACCAGCAACAGAA
CAAAGCTGGATGGAGAATGATTTTGACGAGCTGAGAGAAGAAGGCTTCAG
ACGATCAAATTACTCTGAGCTACGGGAGGACATTCAAACCAAAGGCAAAG
AAGTTGAAAACTTTGAAAAAAATTTAGAAGAATGTATAACTAGAATAACC
AATACAGAGAAGTGCTTAAAGGAGCTGATGGAGCTGAAAACCAAGGCTCG
AGAACTACGTGAAGAATGCAGAAGCCTCAGGAGCCGATGCGATCAACTGG
AAGAAAGGGTATCAGCAATGGAAGATGAAATGAATGAAATGAAGCGAGAA
GGGAAGTTTAGAGAAAAAAGAATAAAAAGAAATGAGCAAAGCCTCCAAGA
AATATGGGACTATGTGAAAAGACCAAATCTACGTCTGATTGGTGTACCTG
AAAGTGATGTGGAGAATGGAACCAAGTTGGAAAACACTCTGCAGGATATT
ATCCAGGAGAACTTCCCCAATCTAGCAAGGCAGGCCAACGTTCAGATTCA
GGAAATACAGAGAACGCCACAAAGATACTCCTCGAGAAGAGCAACTCCAA
GACACATAATTGTCAGATTCACCAAAGTTGAAATGAAGGAAAAAATGTTA
AGGGCAGCCAGAGAGAAAGGTCGGGTTACCCTCAAGGAAAGCCCATCAGA
CTAACAGCGGATCTCTCGGCAGAAACCCTACAAGCCAGAAGAGAGTGGGG
GCCAATATTCAACATTCTTAAAGAAAAGAATTTTCAACCCAGAATTTCAT
ATCCAGCCAAACTAAGCTTCATAAGTGAAGGAGAAATAAAATACTTTATA
GACAAGCAAATGTTGAGAGATTTTGTCACCACCAGGCCTGCCCTAAAAGA
GCTCCTGAAGGAAGCGCTAAACATGGAAAGGAACAACCGGTACCAGCCGC
TGCAAAATCATGCCAAAATGTAAAGACCATCGAGACTAGGAAGAAACTGC
ATCAACTAATGAGCAAAATCACCAGCTAACATCATAATGACAGGATCAAA
TTCACACATAACAATATTAACTTTAAATATAAATGGACTAAATTCTGCAA
TTAAAAGACACAGACTGGCAAGTTGGATAAAGAGTCAAGACCCATCAGTG
TGCTGTATTCAGGAAACCCATCTCACGTGCAGAGACACACATAGGCTCAA
AATAAAAGGATGGAGGAAGATCTACCAAGCCAATGGAAAACAAAAAAAGG
CAGGGGTTGCAATCCTAGTCTCTGATAAAACAGACTTTAAACCAACAAAG
ATCAAAAGAGACAAAGAAGGCCATTACATAATGGTAAAGGGATCAATTCA
ACAAGAGGAGCTAACTATCCTAAATATTTATGCACCCAATACAGGAGCAC
CCAGATTCATAAAGCAAGTCCTCAGTGACCTACAAAGAGACTTAGACTCC
CACACATTAATAATGGGAGACTTTAACACCCCACTGTCAACATTAGACAG
ATCAACGAGACAGAAAGTCAACAAGGATACCCAGGAATTGAACTCAGCTC
TGCACCAAGCAGACCTAATAGACATCTACAGAACTCTCCACCCCAAATCA
ACAGAATATACATTTTTTTCAGCACCACACCACACCTATTCCAAAATTGA
CCACATAGTTGGAAGTAAAGCTCTCCTCAGCAAATGTAAAAGAACAGAAA
TTATAACAAACTATCTCTCAGACCACAGTGCAATCAAACTAGAACTCAGG
ATTAAGAATCTCACTCAAAGCCGCTCAACTACATGGAAACTGAACAACCT
GCTCCTGAATGACTACTGGGTACATAACGAAATGAAGGCAGAAATAAAGA
TGTTCTTTGAAACCAACGAGAACAAAGACACCACATACCAGAATCTCTGG
GACGCATTCAAAGCAGTGTGTAGAGGGAAATTTATAGCACTAAATGCCTA
CAAGAGAAAGCAGGAAAGATCCAAAATTGACACCCTAACATCACAATTAA
AAGAACTAGAAAAGCAAGAGCAAACACATTCAAAAGCTAGCAGAAGGCAA
GAAATAACTAAAATCAGAGCAGAACTGAAGGAAATAGAGACACAAAAAAC
CCTTCAAAAAATCAATGAATCCAGGAGCTGGTTTTTTGAAAGGATCAACA
AATTGATAGACCGCTAGCAAGACTAATAAAGAAAAAAAGAGAGAAGAATC
AAATAGACACAATAAAAAATGATAAAGGGGATATCACCACCGATCCCACA
GAAATACAAACTACCATCAGAGAATACTACAAACACCTCTACGCAAATAA
ACTAGAAAATCTAGAAGAAATGGATACATTCCTCGACACATACACTCTCC
CAAGACTAAACCAGGAAGAAGTTGAATCTCTGAATCGACCAATAACAGGC
TCTGAAATTGTGGCAATAATCAATAGTTTACCAACCAAAAAGAGTCCAGG
ACCAGATGGATTCACAGCCGAATTCTACCAGAGGTACAAGGAGGAACTGG
TACCATTCCTTCTGAAACTATTCCAATCAATAGAAAAAGAGGGAATCCTC
CCTAACTCATTTTATGAGGCCAGCATCATTCTGATACCAAAGCCGGGCAG
AGACACAACCAAAAAAGAGAATTTTAGACCAATATCCTTGATGAACATTG
ATGCAAAAATCCTCAATAAAATACTGGCAAACCGAATCCAGCAGCACATC
AAAAAGCTTATCCACCATGATCAAGTGGGCTTCATCCCTGGGATGCAAGG
CTGGTTCAATATACGCAAATCAATAAATGTAATCCAGCATATAAACAGAG
CCAAAGACAAAAACCACATGATTATCTCAATAGATGCAGAAAAAGCCTTT
GACAAAATTCAACAACCCTTCATGCTAAAAACCTCAATAAATTAGGTATT
GATGGGACGTATTTCAAAATAATAAGAGCTATCTATGACAAACCCACAGC
CAATATCATACTGAATGGGCAAAAACTGGAAGCATTCCCTTTGAAAACCG
GCACAAGACAGGGATGCCCTCTCTCACCGCTCCTATTCAACATAGTGTTG
GAAGTTCTGGCCAGGGCAATCAGGCAGGAGAAGGAAATAAAGGGTATTCA
ATTAGGAAAAGAGGAAGTCAAATTGTCCCTGTTTGCAGACGACATGATTG
TTTATCTAGAAAACCCCATCGTCTCAGCCCAAAATCTCCTTAAGCTGATA
AGCAACTTCAGCAAAGTCTCAGGATACAAAATCAATGTACAAAAATCACA
AGCATTCTTATACACCAACAACAGACAAACAGAGAGCCAAATCATGGGTG
AACTCCCATTCACAATTGCTCAAAGAGAATAAAATACCTAGGAATCCAAC
TTACAAGGGATGTGAAGGACCTCTTCAAGGAGAACTACAAACCACTGCTC
AAGGAAATAAAAGAGGAGACAAACAAATGGAAGAACATTCCATGCTCATG
GGTAGGAAGAATCAATATCGTGAAAATGGCCATACTGCCCAAGGTAATTT
ACAGATTCAATGCCATCCCCATCAAGCTACCAATGACTTTTCTTCACAGA
ATTGGAAAAAACTACTTTAAAGTTCATATGGAACCAAAAAAGAGCCCGCA
TTGCCAAGTCAATCCTAAGCCAAAAGAACAAAGCTGGAGGCATCACACTA
CCTGACTTCAAACTATACTACAAGGCTACAGTAACCAAAACAGCATGGTA
CTGGTACCAAAACAGAGATATAGATCAATGGAACAGAACAGAGCCCTCAG
AAATAATGCCGCATATCTACAACTATCTGATCTTTGACAAACCTGAGAAA
AACAAGCAATGGGGAAAGGATTCCCTATTAATAAATGGTGCTGGGAAAAC
TGGCTAGCCATATGTAGAAAGCTGAAACTGGATCCCTTCCTTACACCTTA
TACAAAAATCAATTCAAGATGGATTAAAGATTTAAACGTTAAACCTAAAA
CCATAAAAACCCTAGAAGAAAACCTAGGCATTACCATTCAGGACATAGGC
GTGGGCAAGGACTCATGTCCAAAACACCAAAAGCAATGGCAACAAAAGAC
AAAATTGACAAATGGGATCTAATTAAACTAAAGAGCTTCTGCACAGCAAA
AGAAACTACCATCAGAGTGAACAGGCAACCTACAACATGGGAGAAAATTT
TTGCAACCTACTCATCTGACAAAGGGCTAATATCCAGAATCTACAATGAA
CTCAAACAAATTTACAAGAAAAAAACAAACAACCCCATCAAAAAGTGGGC
GAAGGACATGAACAGACACTTCTCAAAAGAAGACATTTATGCAGCCAAAA
AACACATGAAGAAATGCTCATCATCACTGGCCATCAGAGAAATGCAAATC
AAAACCACTATGAGATATCATCTCACACCAGTTAGAATGGCAATCATTAA
AAAGTCAGGAAACAACAGGTGCTGGAGAGGATGCGGAGAAATAGGAACAC
TTTTACACTGTTGGTGGGACTGTAAACTAGTTCAACCATTGTGGAAGTCA
GTGTGGCGATTCCTCAGGGATCTAGAACTAGAAATACCATTTGACCCAGC
CATCCCATTACTGGGTATATACCCAAATGAGTATAAATCATGCTGCTATA
AAGACACATGCACACGTATGTTTATTGCGGCACTATTCACAATAGCAAAG
ACTTGGAACCAACCCAAATGTCCAACAATGATAGACTGGATTAAGAAAAT
GTGGCACATATACACCATGGAATACTATGCAGCCATAAAAAATGATGAGT
TCATATCCTTTGTAGGGACATGGATGAAATTGGAAACCATCATTCTCAGT
AAACTATCGCAAGAACAAAAAACCAAACACCGCATATTCTCACTCATAGG
TGGGAATTGAACAATGAGATCACATGGACACAGGAAGGGGAATATCACAC
TCTGGGGACTGTGGTGGGGTCGGGGGAGGGGGGAGGGATAGCATTGGGAG
ATATACCTAATGCTAGATGACACATTAGTGGGTGCAGCGCACCAGCATGG
CACATGTATACATATGTAACTAACCTGCACAATGTGCACATGTACCCTAA
AACTTAGAGTATAATAAA-3′
SEQ ID NO 37
(b) Hot element consensus
6,021 nucleotides
5′-GGGGGAGGAGCCAAGATGGCCGAATAGGAACAGCTCCGGTCTACAGC
TCCCAGCGTGAGCGACGCAGAAGACGGTGATTTCTGCATTTCCATCTGAG
GTACCGGGTTCATCTCACTAGGGAGTGCCAGACAGTGGGCGCAGGCCAGT
GTGTGTGCGCACCGTGCGCGAGCCGAAGCAGGGCGAGGCATTGCCTCACC
TGGGAAGCGCAAGGGGTCAGGGAGTTCCCTTTCCGAGTCAAAGAAAGGGG
TGACGGACGCACCTGGAAAATCGGGTCACTCCCACCCGAATATTGCGCTT
TTCAGACCGGCTTAAGAAACGGCGCACCACGAGACTATATCCCACACCTG
GCTCGGAGGGTCCTACGCCCACGGAATCTCGCTGATTGCTAGCACAGCAG
TCTGAGATCAAACTGCAAGGCGGCAACGAGGCTGGGGGAGGGGCGCCCGC
CATTGCCCAGGCTTGCTTAGGTAAACAAAGCAGCCGGGAAGCTCGAACTG
GGTGGAGCCCACCACAGCTCAAGGAGGCCTGCCTGCCTCTGTAGGCTCCA
CCTCTGGGGGCAGGGCACAGACAAACAAAAAGACAGCAGTAACCTCTGCA
GACTTAAGTGTCCCTGTCTGACAGCTTTGAAGAGAGCAGTGGTTCTCCCA
GCACGCAGCTGGAGATCTGAGAACGGGCAGACTGCCTCCTCAAGTGGGTC
GCTGACTCCTGACCCCCGAGCAGCCTAACTGGGAGGCACCCCCCAGCAGG
GGCACACTGACACCTCACACGGCAGGGTATTCCAACAGACCTGCAGCTGA
GGGTCCTGTCTGTTAGAAGGAAAACTAACAACCAGAAAGGACATCTACAC
CGAAAACCCATCTGTACATCACCATCATCAAAGACCAAAAGTAGATAAAA
CCACAAAGATGGGGAAAAAACAGAACAGAAAAACTGGAAACTCTAAAACG
CAGAGCGCCTCTCCTCCTCCAAAGGAACGCAGTTCCTCACCAGCAACAGA
ACAAAGCTGGATGGAGAATGATTTTGATGAGCTGAGAGAAGAAGGCTTCA
GACGATCAAATTACTCTGAGCTACGGGAGGACATTCAAACCAAAGGCAAA
GAAGTTGAAAACTTTGAAAAAAATTTAGAAGAATGTATAACTAGAATAAC
CAATACAGAGAAGTGCTTAAAGGAGCTGATGGAGCTGAAAACCAAGGCTC
GAGAACTACGTGAAGAATGCAGAAGCCTCAGGAGGCGATGCGATCAACTG
GAAGAAAGGGTATCAGCAATGGAAGATGAAATGAATGAAATGAAGCGAGA
AGGGAAGTTTAGAGAAAAAAGAATAAAAAGAAATGAGCAAAGCCTCCAAG
AAATATGGGACTATGTGAAAAGACCAAATCTACGTCTGATTGGTGTACCT
GAAAGTGATGTGGAGAATGGAACCAAGTTGGAAAACACTCTGCAGGATAT
TATCCAGGAGAACTTCCCCAATCTAGCAAGGCAGGCCAACGTTCAGATTC
AGGAAATACAGAGAACGCCACAAAGATACTCCTCGAGAAGAGCAACTCCA
AGACACATAATTGTCAGATTCACCAAAGTTGAAATGAAGGAAAAAATGTT
AAGGGCAGCCAGAGAGAAAGGTCGGGTTACCCTCAAAGGAAAGCCCATCA
GACTAACAGCGGATCTGTCGGCAGAAACCCTACAAGCCAGAAGAGAGTGG
GGGCCAATATTCAACATTCTAAAGAAAAGAATTTTCAACCCAGAATTTCA
TATCCAGCCAAACTAAGCTTCATAAGTGAAGGAGAAATAAAATACTTTAT
AGACAAGCAAATGTTGAGAGATTTTGTCACCACCAGGCCTGCCCTAAAAG
AGCTCCTGAAGGAAGCGCTAAACATGGAAAGGAACAACCGGTACCAGCCG
CTGCAAAATCATGCCAAAATGTAAAGACCATCGAGACTAGGAAGAAACTG
CATCAACTAATGAGCAAAATCACCAGCTAACATCATAATGACAGGATCAA
ATTCACACATAACAATATTAACTTTAAATATAAATGGACTAAATTCTGCA
ATTAAAAGACACAGACTGGCAAGTTGGATAAAGAGTCAAGACCCATCAGT
GTGCTGTATTCAGGAAACCCATCTCACGTGCAGAGACACACATAGGCTCA
AAATAAAAGGATGGAGGAAGATCTACCAAGCCAATGGAAAACAAAAAAAG
GCAGGGGTTGCAATCCTAGTCTCTGATAAAACAGACTTTAAACCAACAAA
GATCAAAAGAGACAAAGAAGGCCATTACATAATGGTAAAGGGATCAATTC
AACAAGAGGAGCTAACTATCCTAAATATTTATGCACCCAATACAGGAGCA
CCCAGATTCATAAAGCAAGTCCTCAGTGACCTACAAAGAGACTTAGACTC
CCACACATTAATAATGGGAGACTTTAACACCCCACTGTCAACATTAGACA
GATCAACGAGACAGAAAGTCAACAAGGATACCCAGGAATTGAACTCAGCT
CTGCACCAAGCAGACCTAATAGACATCTACAGAACTCTCCACCCCAAATC
AACAGAATATACATTTTTTTCAGCACCACACCACACCTATTCCAAAATTG
ACCACATAGTTGGAAGTAAAGCTCTCCTCAGCAAATGTAAAAGAACAGAA
ATTATAACAAACTATCTCTCAGACCACAGTGCAATCAAACTAGAACTCAG
GATTAAGAATCTCACTCAAAGCCGCTCAACTACATGGAAACTGAACAACC
TGCTCCTGAATGACTACTGGGTACATAACGAAATGAAGGCAGAAATAAAG
ATGTTCTTTGAAACCAACGAGAACAAAGACACCACATACCAGAATCTCTG
GGACGCATTCAAAGCAGTGTGTAGAGGGAAATTTATAGCACTAAATGCCT
ACAAGAGAAAGCAGGAAAGATCCAAAATTGACACCCTAACATCACAATTA
AAAGAACTAGAAAAGCAAGAGCAAACACATTCAAAAGCTAGCAGAAGGCA
AGAAATAACTAAAATCAGAGCAGAACTGAAGGAAATAGAGACACAAAAAA
CCCTTCAAAAAATCAATGAATCCAGGAGCTGGTTTTTTGAAAGGATCAAC
AAAATTGATAGACCGCTAGCAAGACTAATAAAGAAAAAAAGAGAGAAGAA
TCAAATAGACACAATAAAAAATGATAAAGGGGATATCACCACCGATCCCA
CAGAAATACAAACTACCATCAGAGAATACTACAAACACCTCTACGCAAAT
AAACTAGAAAATCTAGAAGAAATGGATACATTCCTCGACACATACACTCT
CCCAAGACTAAACCAGGAAGAAGTTGAATCTCTGAATCGACCAATAACAG
GCTCTGAAATTGTGGCAATAATCAATAGTTTACCAACCAAAAAGAGTCCA
GGACCAGATGGATTCACAGCCGAATTCTACCAGAGGTACAAGGAGGAACT
GGTACCATTCCTTCTGAAACTATTCCAATCAATAGAAAAAGAGGGAATCC
TCCCTAACTCATTTTATGAGGCCAGCATCATTCTGATACCAAAGCCGGGC
AGAGACACAACCAAAAAGAGAATTTTAGACCAATATCCTTGATGAACATT
GATGCAAAAATCCTCAATAAAATACTGGCAAACCGAATCCAGCAGCACAT
CAAAAAGCTTATCCACCATGATCAAGTGGGCTTCATCCCTGGGATGCAAG
GCTGGTTCAATATACGCAAATCAATAAATGTAATCCAGCATATAAACAGA
GCCAAAGACAAAAACCACATGATTATCTCAATAGATGCAGAAAAAGCCTT
TGACAAAATTCAACAACCCTTCATGCTAAAAACTCTCAATAAATTAGGTA
TTGATGGGACGTATTTCAAAATAATAAGAGCTATCTATGACAAACCCACA
GCCAATATCATACTGAATGGGCAAAAACTGGAAGCATTCCCTTTGAAAAC
CGGCACAAGACAGGGATGCCCTCTCTCACCGCTCCTATTCAACATAGTGT
TGGAAGTTCTGGCCAGGGCAATCAGGCAGGAGAAGGAAATAAAGGGTATT
CAATTAGGAAAAGAGGAAGTCAAATTGTCCCTGTTTGCAGACGACATGAT
TGTTTATCTAGAAAACCCCATCGTCTCAGCCCAAAATCTCCTTAAGCTGA
TAAGCAACTTCAGCAAAGTCTCAGGATACAAAATCAATGTACAAAAATCA
CAAGCATTCTTATACACCAACAACAGACAAACAGAGAGCCAAATCATGGG
TGAACTCCCATTCACAATTGCTTCAAAGAGAATAAAATACCTAGGAATCC
AACTTACAAGGGATGTGAAGGACCTCTTCAAGGAGAACTACAAACCACTG
CTCAAGGAAATAAAAGAGGAGACAAACAAATGGAAGAACATTCCATGCTC
ATGGGTAGGAAGAATCAATATGGTGAAAATGGCCATACTGCCCAAGGTAA
TTTACAGATTCAATGCCATCCCCATCAAGCTACCAAGACTTTCTTCACAG
AATTGGAAAAAACTACTTTAAAGTTCATATGGAACCAAAAAAGAGCCCGC
ATTGCCAAGTCAATCCTAAGCCAAAAGAACAAAGCTGGAGGCATCACACT
ACCTGACTTCAAACTATACTACAAGGCTACAGTAACCAAAACAGCATGGT
ACTGGTACCAAAACAGAGATATAGATCAATGGAACAGAACAGAGCCCTCA
GAAATAATGCCGCATATCTACAACTATGTGATCTTTGACAAACCTGAGAA
AAACAAGCAATGGGGAAAGGATTCCCTATTTAATAAATGGTGCTGGGAAA
ACTGGCTAGCCATATGTAGAAAGCTGAAACTGGATCCCTTCCTTACACCT
TATACAAAAATCAATTCAAGATGGATTAAAGATTTAAACGTTAAACCTAA
AACCATAAAAACCCTAGAAGAAAACCTAGGCATTACCATTCAGGACATAG
GCGTGGGCAAGGACTTCATGTCCAAAACACCAAAAGCAATGGCAACAAAA
GACAAAATTGACAAATGGGATCTAATTAAACTAAAGAGCTTCTGCACAGC
AAAAGAAACTACCATCAGAGTGAACAGGCAACCTACAACATGGGAGAAAA
TTTTTGCAACCTACTCATCTGACAAAGGGCTAATATCCAGAATCTACAAT
GAACTCAAACAAATTTACAAGAAAAAAACAAACAACCCCATCAAAAAGTG
GGCGAAGGACATGAACAGACACTTCTCAAAAGAAGACATTTATGCAGCCA
AAAAACACATGAAGAAATGCTCATCATCACTGGCCATCAGAGAAATGCAA
ATCAAAACCACTATGAGATATCATCTCACACCAGTTAGAATGGCAATCAT
TAAAAAGTCAGGAAACAACAGGTGCTGGAGAGGATGCGGAGAAATAGGAA
CACTTTTACACTGTTGGTGGGACTGTAAACTAGTTCAACCATTGTGGAAG
TCAGTGTGGCGATTCCTCAGGGATCTAGAACTAGAAATACCATTTGACCC
AGCCATCCCATTACTGGGTATATACCCAAATGAGTATAAATCATGCTGCT
ATAAAGACACATGCACACGTATGTTTATTGCGGCACTATTCACAATAGCA
AAGACTTGGAACCAACCCAAATGTCCAACAATGATAGACTGGATTAAGAA
AATGTGGCACATATACACCATGGAATACTATGCAGCCATAAAAAATGATG
AGTTCATATCCTTTGTAGGGACATGGATGAAATTGGAAACCATCATTCTC
AGTAAACTATCGCAAGAACAAAAAACCAAACACCGCATATTCTCACTCAT
AGGTGGGAATTGAACAATGAGATCACATGGACACAGGAAGGGGAATATCA
CACTCTGGGGACTGTGGTGGGGTCGGGGGAGGGGGGAGGGATAGCATTGG
GAGATATACCTAATGCTAGATGACACATTAGTGGGTGCAGCGCACCAGCA
TGGCACATGTATACATATGTAACTAACCTGCACAATGTGCACATGTACCC
TAAAACTTAGAGTATAATAAA-3′
SEQ ID NO 38
(c) 90-clement consensus
6,022 nucleotides
5′-GGGGGAGGAGCCAAGATGGCCGAATAGGAACAGCTCCGGTCTACAGC
TCCCAGCGTGAGCGACGCAGAAGACGGGTGATTTCTGCATTTCCATCTGA
GGTACCGGGTTCATCTCACTAGGGAGTGCCAGACAGTGGGCGCAGGCCAG
TGTGTGTGCGCACCGTGCGCGAGCCGAAGCAGGGCGAGGCATTGCCTCAC
CTGGGAAGCGCAAGGGGTCAGGGAGTTCCCTTTCCGAGTCAAAGAAAGGG
GTGACGGACGCACCTGGAAAATCGGGTCACTCCCACCCGAATATTGCGCT
TTTCAGACCGGCTTAAGAAACGGCGCACCACGAGACTATATCCCACACCT
GGCTCAGAGGGTCCTACGCCCACGGAATCTCGCTGATTGCTAGCACAGCA
GTCTGAGATCAAACTGCAAGGCGGCAACGAGGCTGGGGGAGGGGCGCCCG
CCATTGCCCAGGCTTGCTTAGGTAAACAAAGCAGCCGGGAAGCTCGAACT
GGGTGGAGCCCACCACAGCTCAAGGAGGCCTGCCTGCCTCTGTAGGCTCC
ACCTCTGGGGGCAGGGCACAGACAAACAAAAAGACAGCAGTAACCTCTGC
AGACTTAAGTGTCCCTGTCTGACAGCTTTGAAGAGAGCAGTGGTTCTCCC
AGCACGCAGCTGGAGATCTGAGAACGGGCAGACTGCCTCCTCAAGTGGGT
CCCTGACCCCTGACCCCCGAGCAGCCTAACTGGGAGGCACCCCCCAGCAG
GGGCACACTGACACCTCACACGGCAGGGTATTCCAACAGACCTGCAGCTG
AGGGTCCTGTCTGTTAGAAGGAAAACTAACAACCAGAAAGGACATCTACA
CCGAAAACCCATCTGTACATGACCATCATCAAAGACCAAAAGTAGATAAA
ACCACAAAGATGGGGAAAAAACAGAACAGAAAAACTGGAAACTCTAAAAC
GCAGAGCGCCTCTCCTCCTCCAAAGGAACGCAGTTCCTCACCAGCAACAG
AACAAAGCTGGATGGAGAATGATTTTGACGAGCTGAGAGAAGAAGGCTTC
AGACGATCAAATTACTCTGAGCTACGGGAGGACATTCAAACCAAAGGCAA
AGAAGTTGAAAACTTTGAAAAAAATTTAGAAGAATGTATAACTAGAATAA
CCAATACAGAGAAGTGCTTAAAGGAGCTGATGGAGCTGAAAACCAAGGCT
CGAGAACTACGTGAAGAATGCAGAAGGCTCAGGAGCCGATGCGATCAACT
GGAAGAAAGGGTATCAGCAATGGAAGATGAAATGAATGAAATGAAGCGAG
AAGGGAAGTTTAGAGAAAAAAGAATAAAAAGAAATGAGCAAAGCCTCCAA
GAAATATGGGACTATGTGAAAAGACCAAATCTACGTCTGATTGGTGTACC
TGAAAGTGATGTGGAGAATGGAACCAAGTTGGAAAACACTCTGCAGGATA
TTATCCAGGAGAACTTCCCCAATCTAGCAAGGCAGGCCAACGTTCAGATT
CAGGAAATACAGAGAACGCCACAAAGATACTCCTCGAGAAGAGCAACTCC
AAGACACATAATTGTCAGATTCACCAAAGTTGAAATGAAGGAAAAAATGT
TAAGGGCAGCCAGAGAGAAAGGTCGGGTTACCCTCAAAGGAAGCCCATCA
GACTAACAGCGGATCTCTCGGCAGACCCTACAAGCCAGAAGAGAGTGGGG
GCCAATATTCAACATTCTTAAAGAAAAGAATTTTCAACCCAGAATTTCAT
ATCCAGCCAAACTAAGCTTCATAAGTGAAGGAGAAATAAAATACTTTATA
GACAAGCAAATGCTGAGAGATTTTGTCACCACCAGGCCTGCCCTAAAAGA
GCTCCTGAAGGAAGCGCTAAACATGGAAAGGAACAACCGGTACCAGCCGC
TGCAAAATCATGCCAAAATGTAAAGACCATCGAGACTAGGAAGAAACTGC
ATCAACTAATGAGCAAAATCACCAGCTAACATCATAATGACAGGATCAAA
TTCACACATAACAATATTAACTTTAAATATAAATGGACTAAATTCTGCAA
TTAAAAGACACAGACTGGCAAGTTGGATAAAGAGTCAAGACCCATCAGTG
TGCTGTATTCAGGAAACCCATCTCACGTGCAGAGACACACATAGGCTCAA
AATAAAAGGATGGAGGAAGATCTACCAAGCCAATGGAAAACAAAAAAAGG
CAGGGGTTGCAATCCTAGTCTCTGATAAAACAGACTTTAAACCAACAAAG
ATCAAAAGAGACAAAGAAGGCCATTACATAATGGTAAAGGGATCAATTCA
ACAAGAGGAGCTAACTATCCTAAATATTTATGCACCCAATACAGGAGCAC
CCAGATTCATAAAGCAAGTCCTCAGTGACCTACAAAGAGACTTAGACTCC
CACACATTAATAATGGGAGACTTTAACACCCCACTGTCAACATTAGACAG
ATCAACGAGACAGAAAGTCAACAAGGATACCCAGGAATTGAACTCAGCTC
TGCACCAAGCAGACCTAATAGACATCTACAGAACTCTCCACCCCAAATCA
ACAGAATATACATTTTTTTCAGCACCACACCACACCTATTCCAAAATTGA
CCACATAGTTGGAAGTAAAGCTCTCCTCAGCAAATGTAAAAGAACAGAAA
TTATAACAAACTATCTCTCAGACCACAGTGCAATCAAACTAGAACTCAGG
ATTAAGAATCTCACTCAAAGCCGCTCAACTACATGGAAACTGAACAACCT
GCTCCTGAATGACTACTGGGTACATAACGAAATGAAGGCAGAAATAAAGA
TGTTCTTTGAAACCAACGAGAACAAAGACACCACATACCAGAATCTCTGG
GACGCATTCAAAGCAGTGTGTAGAGGGAAATTTATAGCACTAAATGCCTA
CAAGAGAAAGCAGGAAAGATCCAAAATTGACACCCTAACATCACAATTAA
AAGAACTAGAAAAGCAAGAGCAAACACATTCAAAAGCTAGCAGAAGGCAA
GAAATAACTAAAATCAGAGCAGAACTGAAGGAAATAGAGACACAAAAAAC
CCTTCAAAAAATCAATGAATCCAGGAGCTGGTTTTTTGAAAGGATCAACA
AAATTGATAGACCGCTAGCAAGACTAATAAAGAAAAAAAGAGAGAAGAAT
CAAATAGACACAATAAAAAATGATAAAGGGGATATCACCACCGATCCCAC
AGAAATACAAACTACCATCAGAGAATACTACAAACACCTCTACGCAAATA
AACTAGAAAATCTAGAAGAAATGGATACATTCCTCGACACATACACTCTC
CCAAAGACTAAACCAGGAAGAAGTTGAATCTCTGAATAGACCAATAACAG
GCTCTGAAATTGTGGCAATAATCAATAGTTTACCAACCAAAAAGAGTCCA
GGACCAGATGGATTCACAGCCGAATTCTACCAGAGGTACAAGGAGGAACT
GGTACCATTCCTTCTGAAACTATTCCAATCAATAGAAAAAGAGGGAATCC
TCCCTAACTCATTTTATGAGGCCAGCATCATTCTGATACCAAAGCCGGGC
AGAGACACAACCAAAAAAGAGAATTTTAGACCAATATCCTTGATGAACAT
TGATGCAAAAATCCTCAATAAAATACTGGCAAACCGAATCCAGCAGCACA
TCAAAAAGCTTATCCACCATGATCAAGTGGGCTTCATGCCTGGGATGCAA
GGCTGGTTCAATATACGCAAATCAATAAATGTAATCCAGCATATAAACAG
AGCCAAAGACAAAAACCACATGATTATCTCAATAGATGCAGAAAAAGCCT
TTGACAAAATTCAACAACCCTTCATGCTAAAAACTCTCAATAAATTAGGT
ATTGATGGGACGTATTTCAAAATAATAAGAGCTATCTATGACAAACCCAC
AGCCAATATCATACTGAATGGGCAAAAACTGGAAGCATTCCCTTTGAAAA
CTGGCACAAGACAGGGATGCCCTCTCTCACCGCTCCTATTCAACATAGTG
TTGGAAGTTCTGGCCAGGGCAATCAGGCAGGAGAAGGAAATAAAGGGTAT
TCAATTAGGAAAAGAGGAAGTCAAATTGTCCCTGTTTGCAGACGACATGA
TTGTTTATCTAGAAAACCCCATCGTCTCAGCCCAAAATCTCCTTAAGCTG
ATAAGCAACTTCAGCAAAGTCTCAGGATACAAAATCAATGTACAAAAATC
ACAAGCATTCTTATACACCAACAACAGACAAACAGAGAGCCAAATCATGG
GTGAACTCCCATTCACAATTGCTTCAAAGAGAATAAAATACCTAGGAATC
CAACTTACAAGGGATGTGAAGGACCTCTTCAAGGAGAACTACAAACCACT
GCTCAAGGAAATAAAAGAGGACACAAACAAATGGAAGAACATTCCATGCT
CATGGGTAGGAAGAATCAATATCGTGAAAATGGCCATACTGCCCAAGGTA
ATTTACAGATTCAATGCCATCCCCATCAAGCTACCAATGACTTTCTTCAC
AGAATTGGAAAAAACTACTTTAAAGTTCATATGGAACCAAAAAAGAGCCC
GCATTGCCAAGTCAATCCTAAGCCAAAAGAACAAAGCTGGAGGCATCACA
CTACCTGACTTCAAACTATACTACAAGGCTACAGTAACCAAAACAGCATG
GTACTGGTACCAAAACAGAGATATAGATCAATGGAACAGAACAGAGCCCT
CAGAAATAATGCCGCATATCTACAACTATCTGATCTTTGACAAACCTGAG
AAAAACAAGCAATGGGGAAAGGATTCCCTATTTAATAAATGGTGCTGGGA
AAACTGGCTAGCCATATGTAGAAAGCTGAAACTGGATCCCTTCCTTACAC
CTTATACAAAAATCAATTCAAGATGGATTAAAGATTTAAACGTTAGACCT
AAAACCATAAAAAACCCTAGAAGAAAACCTAGGCATTACCATTCAGGACA
TAGGCGTGGGCAAGGACTTCATGTCCAAAACACCAAAAGCAATGGCAACA
AAAGCCAAAATTGACAAATGGGATCTAATTAAACTAAAGAGCTTCTGCAC
AGCAAAAGAAACTACCATCAGAGTGAACAGGCAACCTACAACATGGGAGA
AAATTTTCGCAACCTACTCATCTGACAAAGGGCTAATATCCAGAATCTAC
AATGAACTCAAACAAATTTACAAGAAAAAAACAAACAACCCCATCAAAAA
GTGGGCGAAGGACATGAACAGACACTTCTCAAAAGAAGACATTTATGCAG
CCAAAAAACACATGAAGAAATGCTCATCATCACTGGCCATCAGAGAAATG
CAAATCAAAACCACTATGAGATATCATCTCACACCAGTTAGAATGGCAAT
CATTAAAAAGTCAGGAAACAACAGGTGCTGGAGAGGATGTGGAGAAATAG
GAACACTTTTACACTGTTGGTGGGACTGTAAACTAGTTCAACCATTGTGG
AAGTCAGTGTGGCGATTCCTCAGGGATCTAGAACTAGAAATACCATTTGA
CCCAGCCATCCCATTACTGGGTATATACCCAAAGGACTATAAATCATGCT
GCTATAAAGACACATGCACACGTATGTTTATTGCGGCACTATTCACAATA
GCAAAGACTTGGAACCAACCCAAATGTCCAACAATGATAGACTGGATTAA
GAAAATGTGGCACATATACACCATGGAATACTATGCAGCCATAAAAAATG
ATGAGTTCATATCCTTTGTAGGGACATGGATGAAATTGGAAACCATCATT
CTCAGTAAACTATCGCAAGAACAAAAAACCAAACACCGCATATTCTCACT
CATAGGTGGGAATTGAACAATGAGATCACATGGACACAGGAAGGGGAATA
TCACACTCTGGGGACTGTGGTGGGGTCGGGGGAGGGGGGAGGGATAGCAT
TGGGAGATATACCTAATGCTAGATGACACATTAGTGGGTGCAGCGCACCA
GCATTGGCACATGTATACATATGTAACTAACCTGCACAATGTGCACATGT
ACCCTAAAACTTAGAGTATAATAAA
SEQ ID NO. 34
5′-AATTCTCCGAACGTGTCACGT-3′
SEQ ID NO. 35
5′-AGCTTAAAAAGAGAACGCCACAAAGATACTCTCTTGAAGTATCTTTG
TGGCGTTCTCGGG-3′
SEQ ID NO. 26
5′-cctattggcg ttactatggg aacatacgtc attattgacg
tcaatgggcg ggggtcgttgggcggtcagc caggcgggcc
atttaccgta agttatgtaa cgcggaactc
catatatgggctatgaacta atgagcccgt aattgattac
tattagcccg ggcaatgtgc acatgtaccctaaaacttaa
agtataataa agacgtcagg gttcgaaatc gataagcttg
gatcccccgacctcgagggg ggaggccggc aaggccggat
ccagacatga taagatacat tgatgagtttggacaaacca
caactagaat gcagtgaaga aaatgcttta tttgtgaaat
ttgtgatgctattgctttat ttgtaaccat tataagctgc
aataaacaag ttaacaacaa caaaaaaaaaaaaaaaaaaa
aaaaaaaaaa aaaaaaaaaa aaaaaaaaaa aaaaaaaaaa
aaaaaaaaaaaaaaaaaaaa aaaaaaaata acttgaagca
aataaacaag acgaccaatt caatgtgctggaacaccgta
agtttgcagt ttctgacata taaaatgtgg aggggaccta
tgttaccctaaacctttcca cccactgctt cagctaaagg
gtcaatgaag aacaagcttc ctatgtatcacgtgacagtt
ttgggattca ggcactggta aaggcaacag cccttaagtg
atccaagatattttctcttt ttcacattgc aattctatct
tggtttatca tggggttctt gtattttatcaacatcccta
ctctgacaga acagacaaaa tgctatagcg atggcatcct
tgtagtgggaagaacagacc acagaccctg agagctatcc
tgggtcatcc tcaggaatgt gtgaaaggtagaaaggatct-3′
SEQ ID NO. 27
5′-ggggggagga gccaagatgg ccgaatagga acagctccgg
tctacagctc ccagcgtgagcgacgcagaa gacggtgatt
tctgcatttc catctgaggt accgggttca
tctcactagggagtgccaga cagtgggcgc aggccagtgt
gtgtgcgcac cgtgcgcgag ccgaagcagggcgaggcatt
gcctcacctg ggaagcgcaa ggggtcaggg agttcccttt
ccgagtcaaagaaaggggtg acggacgcac ctggaaaatc
gggtcactcc cacccgaata ttgcgcttttcagaccggct
taagaaacgg cgcaccacga gactatatcc cacacctggc
tcggagggtcctacgcccac ggaatctcgc tgattgctag
cacagcagtc tgagatcaaa ctgcaaggcggcaacgaggc
tgggggaggg gcgcccgcca ttgcccaggc ttgcttaggt
aaacaaagcagcagggaagc tcgaactggg tggagcccac
cacagctcaa ggaggcctgc ctgcctctgtaggctccacc
tctgggggca gggcacagac aaacaaaaag acagcagtaa
cctctgcagacttaagtgtc cctgtctgac agctttgaag
agagcagtgg ttctcccagc acgcagctggagatctgaga
acgggcagac tgcctcctca agtgggtccc tgacccctga
cccccgagcagcctaactgg gaggcacccc ccagcagggg
cacactgaca cctcacacgg cagggtattccaacagacct
gcagctgagg gtcctgtctg ttagaaggaa aactaacaac
cagaaaggacatctacaccg aaaacccatc tgtacatcac
catcatcaaa gaccaaaagt agataaaaccacaaagatgg
ggaaaaaaca gaacagaaaa actggaaact ctaaaacgca
gagcgcctctcctcctccaa aggaacgcag ttcctcacca
gcaacagaac aaagctggat ggagaatgattttgatgagc
tgagagaaga aggcttcaga cgatcaaatt actctgagct
acgggaggacattcaaacca aaggcaaaga agttgaaaac
tttgaaaaaa atttagaaga atgtataactagaataacca
atacagagaa gtgcttaaag gagctgatgg agctgaaaac
caaggctcgagaactacgtg aagaatgcag aagcctcagg
agccgatgcg atcaactgga agaaagggtatcagcaatgg
aagatgaaat gaatgaaatg aagcgagaag ggaagtttag
agaaaaaagaataaaaagaa atgagcaaag cctccaagaa
atatgggact atgtgaaaag accaaatctacgtctgattg
gtgtacctga aagtgatgtg gagaatggaa ccaagttgga
aaacactctgcaggatatta tccaggagaa cttccccaat
ctagcaaggc aggccaacgt tcagattcaggaaatacaga
gaacgccaca aagatactcc tcgagaagag caactccaag
acacataattgtcagattca ccaaagttga aatgaaggaa
aaaatgttaa gggcagccag agagaaaggtcgggttaccc
tcaaaggaaa gcccatcaga ctaacagcgg atctctcggc
agaaaccctacaagccagaa gagagtgggg gccaatattc
aacattctta aagaaaagaa ttttcaacccagaatttcat
atccagccaa actaagcttc ataagtgaag gagaaataaa
atactttatagacaagcaaa tgttgagaga ttttgtcacc
accaggcctg ccctaaaaga gctcctgaaggaagcgctaa
acatggaaag gaacaaccgg taccagccgc tgcaaaatca
tgccaaaatgtaaagaccat caagactagg aagaaactgc
atcaactaat gagcaaaatc accagctaacatcataatga
caggatcaac ttcacacata acaatattaa ctttaaatat
aaatggactaaattctgcaa ttaaaagaca cagactggca
agttggataa agagtcaaga cccatcagtgtgctgtattc
aggaaaccca tctcacgtgc agagacacac ataggctcaa
aataaaaggatggaggaaga tctaccaagc caatggaaaa
caaaaaaagg caggggttgc aatcctagtctctgataaaa
cagactttaa accaacaaag atcaaaagag acaaagaagg
ccattacataatggtaaagg gatcaattca acaagaggag
ctaactatcc taaatattta tgcacccaatacaggagcac
ccagattcat aaagcaagtc ctcagtgacc tacaaagaga
cttagactcccacacattaa taatgggaga ctttaacacc
ccactgtcaa cattagacag atcaacgagacagaaagtca
acaaggatac ccaggaattg aactcagctc tgcaccaagc
agacctaatagacatctaca gaactctcca ccccaaatca
acagaatata catttttttc agcaccacaccacacctatt
ccaaaattga ccacatagtt ggaagtaaag ctctcctcag
caaatgtaaaagaacagaaa ttataacaaa ctatctctca
gaccacagtg caatcaaact agaactcaggattaagaatc
tcactcaaag ccgctcaact acatggaaac tgaacaacct
gctcctgaatgactactggg tacataacga aatgaaggca
gaaataaaga tgttctttga aaccaacgagaacaaagaca
ccacatacca gaatctctgg gacgcattca aagcagtgtg
tagagggaaatttatagcac taaatgccta caagagaaag
caggaaagat ccaaaattga caccctaacatcacaattaa
aagaactaga aaagcaagag caaacacatt caaaagctag
cagaaggcaagaaataacta aaatcagagc agaactgaag
gaaatagaga cacaaaaaac ccttcaaaaaatcaatgaat
ccaggagctg gttttttgaa aggatcaaca aaattgatag
accgctagcaagactaataa agaaaaaaag agagaagaat
caaatagaca caataaaaaa tgataaaggggatatcacca
ccgatcccac agaaatacaa actaccatca gagaatacta
caaacacctctacgcaaata aactagaaaa tctagaagaa
atggatacat tcctcgacac atacactctcccaagactaa
accaggaaga agttgaatct ctgaatcgac caataacagg
ctctgaaattgtggcaataa tcaatagttt accaaccaaa
aagagtccag gaccagatgg attcacagccgaattctacc
agaggtacaa ggaggaactg gtaccattcc ttctgaaact
attccaatcaatagaaaaag agggaatcct ccctaactca
ttttatgagg ccagcatcat tctgataccaaagccgggca
gagacacaac caaaaaagag aattttagac caatatcctt
gatgaacattgatgcaaaaa tcctcaataa aatactggca
aaccgaatcc agcagcacat caaaaagcttatccaccatg
atcaagtggg cttcatccct gggatgcaag gctggttcaa
tatacgcaaatcaataaatg taatccagca tataaacaga
gccaaagaca aaaaccacat gattatctcaatagatgcag
aaaaagcctt tgacaaaatt caacaaccct tcatgctaaa
aactctcaataaattaggta ttgatgggac gtatttcaaa
ataataagag ctatctatga caaacccacagccaatatca
tactgaatgg gcaaaaactg gaagcattcc ctttgaaaac
cggcacaagacagggatgcc ctctctcacc gctcctattc
aacatagtgt tggaagttct ggccagggcaatcaggcagg
agaaggaaat aaagggtatt caattaggaa aagaggaagt
caaattgtccctgtttgcag acgacatgat tgtttatcta
gaaaacccca tcgtctcagc ccaaaatctccttaagctga
taagcaactt cagcaaagtc tcaggataca aaatcaatgt
acaaaaatcacaagcattct tatacaccaa caacagacaa
acagagagcc aaatcatggg tgaactcccattcacaattg
cttcaaagag aataaaatac ctaggaatcc aacttacaag
ggatgtgaaggacctcttca aggagaacta caaaccactg
ctcaaggaaa taaaagagga gacaaacaaatggaagaaca
ttccatgctc atgggtagga agaatcaata tcgtgaaaat
ggccatactgcccaaggtaa tttacagatt caatgccatc
cccatcaagc taccaatgac tttcttcacagaattggaaa
aaactacttt aaagttcata tggaaccaaa aaagagcccg
cattgccaagtcaatcctaa gccaaaagaa caaagctgga
ggcatcacac tacctgactt caaactatactacaaggcta
cagtaaccaa aacagcatgg tactggtacc aaaacagaga
tatagatcaatggaacagaa cagagccctc agaaataatg
ccgcatatct acaactatct gatctttgacaaacctgaga
aaaacaagca atggggaaag gattccctat ttaataaatg
gtgctgggaaaactggctag ccatatgtag aaagctgaaa
ctggatccct tccttacacc ttatacaaaaatcaattcaa
gatggattaa agatttaaac gttaaaccta aaaccataaa
aaccctagaagaaaacctag gcattaccat tcaggacata
ggcgtgggca aggacttcat gtccaaaacaccaaaagcaa
tggcaacaaa agacaaaatt gacaaatggg atctaattaa
actaaagagcttctgcacag caaaagaaac taccatcaga
gtgaacaggc aacctacaac atgggagaaaatttttgcaa
cctactcatc tgacaaaggg ctaatatcca gaatctacaa
tgaactcaaacaaatttaca agaaaaaaac aaacaacccc
atcaaaaagt gggcgaagga catgaacagacacttctcaa
aagaagacat ttatgcagcc aaaaaacaca tgaagaaatg
ctcatcatcactggccatca gagaaatgca aatcaaaacc
actatgagat atcatctcac accagttagaatggcaatca
ttaaaaagtc aggaaacaac aggtgctgga gaggatgcgg
agaaataggaacacttttac actgttggtg ggactgtaaa
ctagttcaac cattgtggaa gtcagtgtggcgattcctca
gggatctaga actagaaata ccatttgacc cagccatccc
attactgggtatatacccaa atgagtataa atcatgctgc
tataaagaca catgcacacg tatgtttattgcggcactat
tcacaatagc aaagacttgg aaccaaccca aatgtccaac
aatgatagactggattaaga aaatgtggca catatacacc
atggaatact atgcagccat aaaaaatgatgagttcatat
cctttgtagg gacatggatg aaattggaaa ccatcattct
cagtaaactatcgcaagaac aaaaaaccaa acaccgcata
ttctcactca taggtgggaa ttgaacaatgagatcacatg
gacacaggaa ggggaatatc acactctggg gactgtggtg
gggtcgggggaggggggagg gatagcattg ggagatatac
ctaatgctag atgacacatt agtgggtgcagcgcaccagc
atggcacatg tatacatatg taactaacct gcacaatgtg
cacatgtaccctaaaactta gagtataat-3′(6019)

1. A method for inhibiting unspecialised proliferation of cancerous tissue in a patient, comprising administering to said patient an interferent RNA (RNAi) or a DNA construct encoding said RNAi, wherein the RNA recognises a portion of at least one LINE-1 (Ll) repeat element.

2. A method for treating a cancerous lesion in a patient, comprising administering to said patient an interferent RNA (RNAi) or a DNA construct encoding said RNAi, wherein the RNA recognises a portion of at least one LINE-1 (Ll) repeat element.

3. A RNAi molecule, wherein the RNA recognises a portion of at least one LINE-1 (L1) repeat element.

4. The RNAi according to claim 3, which is specific for a transcribed open reading frame of a LINE-1 family member.

5. The RNAi according to claim 4, wherein the open reading frame encodes Reverse Transcriptase.

6. The RNAi according to claim 3, wherein the Ll element is an active Ll element.

7. The RNAi according to claim 3, which is specific for a LINE-1 consensus sequence.

8. The RNAi according to claim 3, wherein the RNAi is a short interfering RNA (siRNA).

9. The RNAi according to claim 3, wherein the RNAi is a double-stranded RNA (dsRNA).

10. The RNAi according to claim 3, wherein the RNAi is a short hairpin RNA, adapted for administration by means of an siRNA expression vector.

11. The RNAi according to claim 3, wherein the RNAi sequence recognises and is capable of binding to RNA obtainable by transcription from an ORF comprised within the target Ll.

12. The RNAi according to claim 11, wherein binding of the RNAi is under stringent conditions, such as in a buffer containing 50% formamide and 6×SSC.

13. The RNAi according to claim 3, wherein the Ll sequence recognised by the RNAi comprises at least a portion of an ORF.

14. The RNAi according to claim 13, wherein the Ll ORF sequence is selected from the group consisting of SEQ ID NOS. 20-25 and DNA sequences corresponding thereto.

15. The RNAi according to claim 3, wherein the RNAi comprises at least 20 consecutive nucleotides from a sequence selected from the group consisting of SEQ ID NOS. 39-44.

16. The RNAi according to claim 3, wherein the RNAi is directed to DNA comprised within positions 907 to 1923 of SEQ ID NO. 27 and/or 1987 to 5814 of SEQ ID NO. 27.

17. The RNAi according to claim 16, wherein the RNAi is capable of inhibiting expression of the proteins according to SEQ ID NO. 45 and/or SEQ ID NO. 46.

18. The RNAi according to claim 17, wherein the RNAi comprises a stretch of RNA that corresponds to an RNA sequence encoding the protein according to SEQ ID NO. 45 and/or SEQ ID NO. 46.

19. The RNAi according to claim 18, wherein the RNAi consists of a 20 or 21 bp stretch of RNA that corresponds to an RNA sequence encoding the protein according to SEQ ID NO. 45 and/or SEQ ID NO. 46.

20. The RNAi according to claim 18, wherein the RNAi has the sequence of SEQ ID NO. 19, or its RNA equivalent, SEQ ID NO. 47.

21. The RNAi according to claim 3, wherein the at least one Ll is polymorphic and/or 6 kbp in length.

22. The RNAi according to claim 3, wherein the at least one Ll is highly active in human beings, a “hot Ll,” having at least ⅓ of the activity of LIRP, SEQ ID NO. 27.

23. The RNAi according to claim 3, wherein the at least one Ll sequence is selected from the group consisting of SEQ ID NOS. 35-38, or homologues having at least 70% sequence homology to said SEQ ID NO. or its corresponding DNA sequence.

24. The RNAi according to claim 3, wherein the at least one LINE-1 element is derived from the ‘transcribed group A’ (Ta) subset of Ll elements or from the pre-Ta subset.

25. The RNAi according to claim 24, wherein the at least one Ll is selected from the group consisting of LRE3, LlRP (NCBI accession number AF148856), and accession numbers ac004200, ac002980, al356438, al512428, ac021017, and all37845, SEQ ID NOS. 26-33, respectively.

26. The RNAi according to claim 25, wherein the at least one Ll is LI_{RP}, SEQ ID NO. 27.

27. The RNAi according to claim 10, wherein the expression vector is of retroviral or adenoviral origin comprising a DNA construct encoding the siRNA.

28. The RNAi according to claim 10, wherein the expression vector is a plasmid comprising a DNA construct encoding the siRNA.

29-31. (canceled)

32. A method for stimulating differentiation of cancerous tissue in a patient, comprising administering to said patient an interferent RNA (RNAi) or a DNA construct encoding said RNAi, wherein the RNA recognises a portion of at least one LINE-1 (LI) repeat element.
